(19) United States
(12) Patent Application Publication
(10) Pub. No.: US 2023/0042485 A1
(43) Pub. Date: Feb. 9, 2023

(54) PROCESS TO IDENTIFY CONSORTIA OF PROBIOTIC STRAINS SUITABLE FOR GLUTEN DEGRADATION

(71) Applicant: Evonik Operations GmbH, Essen (DE)

(72) Inventors: Bodo SPECKMANN, Kahl (DE); Michael SCHWARM, Alzenau (DE); Stefan PELZER, Guetersloh (DE); Thomas BERNGRUBER, Hamm (DE); Marco GOBBETTI, Bozen-Bolzano (IT); Raffaella DI CAGNO, Bozen-Bolzano (IT)

(73) Assignee: Evonik Operations GmbH, Essen (DE)

(21) Appl. No.: 17/788,224

(22) PCT Filed: Nov. 27, 2020
(86) PCT No.: PCT/EP2020/083746

§ 371 Date: Jun. 22, 2022

(30) Foreign Application Priority Data
Dec. 23, 2019 (EP) 19219257.3

(51) Int. Cl. C12Q 1/04; A61K 35/741; C12Q 1/37; C12N 1/20; G01N 33/68

(52) U.S. Cl. CPC C12Q 1/04; A61K 35/741; C12Q 1/37; C12N 1/20; G01N 33/68; C12R 2001/01

(57) ABSTRACT

This invention concerns a process to identify consortia of probiotic strains belonging to e.g. the genera Lactobacillus, Bacillus, Pediococcus, and Weissella that can be used in preparations for food supplement, food production, and pharmaceutical applications with the intention to execute a safe and rapid degradation of gluten to non-toxic, non-immunogenic digests.

This invention concerns a process to identify consortia of probiotic strains belonging to e.g. the genera Lactobacillus, Bacillus, Pediococcus, and Weissella that can be used in preparations for food or pet food supplement, food or pet food production, and pharmaceutical applications with the intention to execute a safe and rapid degradation of gluten to non-toxic, non-immunogenic digests.

Gluten is the main protein network of cereals such as wheat, rye, oat, and barley. Gluten includes monomeric α-gliadins, γ-gliadins, Ω-gliadins, which carry peptide sequences with immunogenic and/or toxic potential (the most prominent examples are listed in table 1).

TABLE 1
Immunogenic gliadin peptides

| Gliadin | Position | Sequence |
|---|---|---|
| α9-gliadin | 57-68 | QLQPFPQPQLPY |
| A-gliadin | 62-75 | PQPQLPYPQPQSFP |
| Y-gliadin | 134-153 | QQLPQPQQPQQSFPQQQRPF |
| α2-gliadin | 57-89 | LQLQPFPQPQLPYPQPQLPYPQPQLPY |
| | | PQPQPF |

Dietary intake of gluten can therefore cause health impairments, when incomplete digestion of gliadins or glutenins releases toxic peptides in susceptible individuals. The spectrum of gluten-related disorders includes celiac disease (CD), wheat allergy (WA), non-celiac gluten sensitivity (NCGS), and gluten-sensitive irritable bowel syndrome [1]. Currently, there is no cure available for these disorders—the only effective solution being avoidance of gluten intake, particularly for people with CD. Interestingly, various other health conditions (e.g. schizophrenia, atopy, fibromyalgia, endometriosis, obesity, non-specific gastrointestinal symptoms) have been suggested to benefit from gluten avoidance [2]. These facts explain the rise of gluten-free diets (GFD); and such practice also extends to a large and increasing number of healthy, symptom-free people. For example, reportedly 33% of the US population wants to avoid gluten, and 41% of an athlete population reported being on a GFD for more than 50% of the time [3]. Practicing a GFD is however associated with challenges and adverse effects, which need to be considered in a risk and benefit evaluation. People with CD need to strictly adhere to a GFD, which is difficult to realize, given that even food products considered or claimed as being gluten-free often contain (trace) amounts of gluten that are above a safe limit of gluten intake (typically <20 ppm for CD patients). To ensure food safety for CD patients and related gluten-associated disorders, reliable and efficient strategies are required to support gluten avoidance or detoxification.

In cases where there is no clear indication to maintain a GFD, i.e. where gluten avoidance is rather a lifestyle choice than a medical necessity, adverse side-effects of this diet need to be considered. A GFD is often imbalanced, e.g. due to the avoidance of cereal products, with micronutrient and fiber deficiencies, alongside an excess of calories and an increased content of sugar and saturated fats found in many gluten-replacement foods [4-6]. Potential harms of a GFD therefore include growth/development retardation for children and adolescents, various malnutrition-associated disorders, hyperlipidemia, hyperglycemia, and coronary artery disease [6]. Moreover, long-term adherence to a GFD can cause intestinal microbiome dysbiosis with subsequent adverse health effects [7].

A key determinant of the intestinal fate of gluten and the physiological response to it is the intestinal microbiota, as has been revealed from experiments with differentially colonized mice [8] and from comparisons of microbiota from CD patients versus healthy individuals [9, 10]. Consequently, several microbiota-targeted approaches have been developed in search for treatment options for gluten-related disorders. These approaches can be categorized into: 1. Oral application of Lactobacillus spp. or Bifidobacterium spp. to correct dysbiosis associated with GFD or gluten-related disorders, 2. Oral application of Lactobacillus spp. or Bifidobacterium spp. as non-specific support for gluten-related disorders via undefined mechanisms, 3. Oral application of Lactobacillus spp. or Bifidobacterium spp. to support the degradation of gluten, 4. Oral application of peptide hydrolases isolated from fungi or bacteria to support the degradation of gluten (“glutenases”). So far, all these attempts have failed to deliver a consistent benefit to people in need thereof. Moreover, the application of peptide hydrolases has been discussed as a possible health risk, as they may cause incomplete digestion of gluten, triggering the release of toxic epitopes, which would exacerbate and not ameliorate gluten toxicity [11]. Efficacy of enzyme treatments for CD patients is also limited by poor proteolytic resistance, and limited extent and duration of enzymatic activity during gastrointestinal transit [12].

Recently, the taxonomic classification of several species of the genus Lactobacillus has been updated, according to Zheng J, Wittouck S, Salvetti E, Cmap Franz H M B, Harris P, Mattarelli P W, O'Toole B, Pot P, Vandamme J, Walter K, Watanabe S, Wuyts G E, Felis M G, Ganzle A and Lebeer S, 2020. A taxonomic note on the genus lactobacillus: description of 23 novel genera, emended description of the genus lactobacillus Beijerinck 1901, and Union of Lactobacillaceae and Leuconostocaceae. International Journal of Systematic and Evolutionary Microbiology. https://doi.org/10.1099/ijsem.0.004107. Of particular relevance in the context of this invention are the following species:

| “Old” denomination | Updated denomination (since 2020) |
|---|---|
| Lactobacillus brevis | Levilactobacillus brevis |
| Lactobacillus casei | Lacticaseibacillus casei |
| Lactobacillus paracasei | Lacticaseibacillus paracasei |
| Lactobacillus plantarum | Lactiplantibacillus plantarum |
| Lactobacillus reuteri | Limosilactobacillus reuteri |
| Lactobacillus sanfranciscensis | Fructilactobacillus sanfranciscensis |

We believe that the lack of benefit from probiotic interventions results from improper selection and blending of probiotic strains. A meaningful and vigorous selection process is the prerequisite to identify consortia of synergistically interacting probiotic bacteria that promote the rapid and complete digestion of gluten. Such process has so far not been described and is the subject of this invention.

Rashmi et al. disclosed four gluten-hydrolyzing Bacillus strains that showed resistance towards pH 2 and bile acids [13]. The digests were however not assessed for their putative immunogenicity and occurrence of immunogenic peptides. Likewise, specific peptidase activities of the strains alone or in combination were not assessed. Gluten-hydrolyzing potential of consortia was not assessed.

Phromraksa et al. isolated nine Bacillus strains from Thai traditional fermented food. The strains were assessed by western blotting for gliadin hydrolysis by using crude bacterial extracts. Neither the digestion products nor the immunogenic potential of the digests were characterized [14].

Clark et al. isolated fifty bacterial strains from pig ileum by selective culturing and screened them for PepN, PepI, and PEP activities (corresponding to parts of step 4) (Journal of Allergy and Clinical Immunology, (February 2011) Vol. 127, No. 2, Supp. SUPPL. 1, pp. AB243. Abstract Number: 942. Meeting Info: 2011 American Academy of Allergy, Asthma and Immunology, AAAAI Annual Meeting. San Francisco, Calif., United States. 18 Mar. 2011-22 Mar. 2011 ISSN: 0091-6749).

Similarly, Fernandez et al. used selective culturing to obtain 150 isolates from human saliva [15]. Strains were assessed for gliadin, tripeptide, and 33-mer hydrolysis.

US2013/0121976 A1 claims a method of selecting strains of lactic acid bacteria for use in treatment of celiac disease comprising the steps of selecting the strains with the capacity of degrading the 33-mer, a 20-mer peptide QQLPQPQQPQQSPFQQQRPF, a 13-mer peptide LGQQQPFPPQQPY, and an 18-mer peptide PQLPYPQPQLPYPQPQPF, and wherein said strains can degrade said peptides at pH values between 4 and 6 and in the presence of lysozyme, pepsin, chymotrypsin, and trypsin.

Francavilla et al. assessed in vitro peptidase activities of Lactobacillus strains, showing activities of up to 10 mU/mg for PepN, 10 mU/mg for PepI, 5 mU/mg for PEP, 25 mU/mg for PepQ for strains of the species Lactobacillus plantarum (Lactiplantibacillus plantarum), Lactobacillus bulgaricus, Lactobacillus rhamnosus, Lactobacillus paracasei (Lacticaseibacillus paracasei), and Lactobacillus casei (Lacticaseibacillus casei) [16]. Combined application of ten of these strains led to hydrolysis of gliadin epitopes listed in Table 1 after 24 hours of incubation. Survival of the strains under gastric and small intestinal conditions was not determined, the effectiveness of these strains on gluten digestion in the gastrointestinal tract of humans can therefore not be predicted.

Herran et al. isolated 27 bacterial strains belonging to the species L. salivarius, L. rhamnosus, L. reuteri (Limosilactobacillus reuteri), L. casei (Lacticaseibacillus casei), L. oris, L. gasseri, L. fermentum, L. crispatus, L. brevis (Levilactobacillus brevis), B. subtilis, B. amyloliquefaciens, B. pumilus, and B. licheniformis from the small intestine of humans that showed proteolytic activity against the 33-mer only after a very long incubation time of 24 hours and not against other peptides [17]. Similarly, weak activity against this epitope was found for other strains of human small intestinal origin, again including the species B. subtilis, B. pumilus, and B. licheniformis [18].

The present invention discloses a process to identify consortia of probiotic strains belonging to the genera Lactobacillus, Bacillus, Pediococcus and Weissella that can be used for promoting a complete and rapid degradation of gluten in e.g. food & pharmaceutical applications.

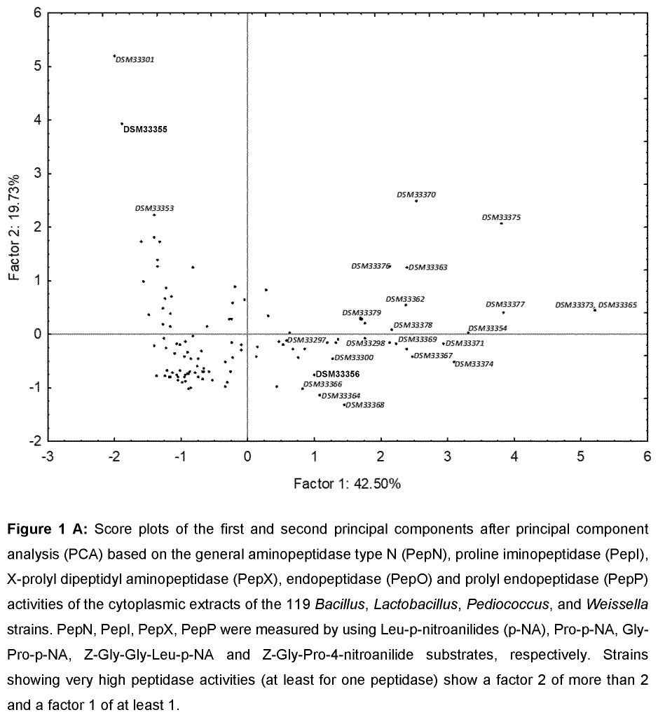

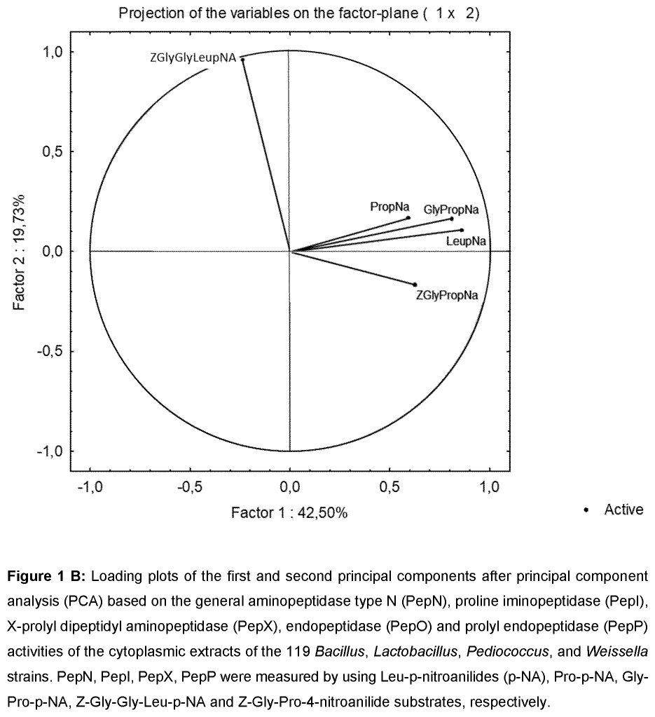

Libraries of bacterial strains derived from gluten-exposed ecological niches (e.g. soil, cereal processing, sourdough, feces, human/animal gastrointestinal tract specimens) are subjected to consecutive screening steps consisting of: resistance to simulated gastrointestinal conditions, adequate protease activity against gluten, adequate peptidase activities against synthetic proline-containing peptide substrates. Strains that have passed these screening steps are combined to consortia (of viable cells or extracts thereof) with complementary peptidase activities and tested for hydrolysis of relevant immunogenic peptides derived from gluten. Consortia that promote rapid and complete removal of these peptides are then applied in gluten hydrolysis experiments under simulated gastrointestinal conditions, thereafter the digests are probed for absence of gluten, gluten-derived immunogenic peptides, and immunogenic potential on duodenal explants from celiac disease patients (see FIG. 1). Finally, consortia are tested in humans in gluten challenge trials with assessment of fecal samples for contents of gluten, gluten-derived immunogenic peptides, and microbiota composition analysis, including contents of introduced strains.

The screening steps disclosed as follows provide a funnel that yields consortia of probiotic strains that can be used for food production (gluten-free food stuffs) as well as dietary supplements and pharma applications (aiding in the safe clearance of gluten in the gut).

The subject of the present invention is therefore a process to identify a consortium of probiotic strains for promoting a degradation of gluten and gluten-derived peptides (epitopes) comprising at least the following steps:
1) Providing a library of at least 10 probiotic bacterial strains;
2) Incubation of the probiotic bacterial strains of step 1) to simulated gastric (pH 1-4) conditions for at least 30 minutes and intestinal conditions (pH 5.5-8.5) for at least 30 minutes and selecting strains with less than 2 log loss of CFU after stimulated gastric and intestinal conditions;
3) Determining proteinase activities of strains selected in step 2) towards gluten and selecting strains with capability to decrease an initial gluten level of at least 5000 ppm by 10 to 70%;
4) Determining activities of peptidases aminopeptidase type N (PepN); PepI, PepO, Prolyl endopeptidyl peptidase (PEP); PepX, and PepQ peptide hydrolase of strains selected in step 3) and selecting strains with peptidase activity of at least 1 U/g for at least one of these peptidases;
5) Combining at least 2 strains selected in step 4) to a consortium of probiotic strains with activities of the peptidases PepN, PepI, PepO, PepX and PepQ of at least 1 U/g for each peptidase;
6) Determining peptidase activities of the consortium of step 5) with peptidase activity towards the 12-mer peptide QLQPFPQPQLPY (Seq-ID No 1), the 14-mer peptide PQPQLPYPQPQSFP (Seq-ID No 2), the 20-mer peptide QQLPQPQQPQQSFPQQQRPF (Seq-ID No 3), and the 33-mer peptide LQLQPFPQPQLPYPQPQLPYPQPQLPYPQPQPF (Seq-ID No 4)) and selection of consortium with a peptidase activity to degrade all four epitopes of more than 50%;
7) Determining peptidase activity for the consortium selected in step 6) for the hydrolysis of gluten with a starting concentration of at least 5000 ppm gluten under simulated gastric (pH 1-4) conditions for at least 30 minutes and intestinal conditions (pH 5.5-8.5) for at least 30 minutes and selecting consortium that reduce an initial gluten level of at least 5000 ppm to a concentration of hydrolyzed and residual gluten of less than 200 ppm.

It is preferred to use more probiotic strains and to screen higher amounts of different strains. Therefore, in an advantageous configuration, the library provided comprises at least 20, preferably at least 30, more preferably at least 40, most preferably at least 50 probiotic strains.

In step 6) it is preferred to select strains with a peptidase activity to degrade all four epitopes of more than 70%, preferably more than 90%.

In a preferred embodiment the enzyme activity of the peptidases aminopeptidase type N (PepN); PepI, PepO, Prolyl endopeptidyl peptidase (PEP); PepX, and PepQ peptide hydrolase is at least 3 U/g (PepP), 5 U/g (PepO), 20 U/g (PepX), 17 U/g (PepI), 20 U/g (PepN) for at least one of these peptidases.

The peptidase activity in step 7) may be determined by e.g. ELISA with appropriate antibodies directed at Pro-rich peptide sequences.

In a preferred configuration, the process further comprises one or more of the following steps:
8) Determining hydrolysis of gluten during wheat bread digestion (1-100 gr of wheat bread) by the mixture of strains selected in step 6) under simulated gastrointestinal conditions and selection of strains with a degradation capacity of the gluten content in wheat bread during 6-24 hours to less than 20 ppm and absence of gluten-derived epitopes (the 12-mer peptide, the 14-mer peptide, the 20-mer peptide and the 33-mer peptide) after 180 min of simulated intestinal digestion;
9) Determining immunogenicity of mixture of strains selected in step 7) by using small intestinal tissue explants from CD patients by determining the expression of the cytokines Interleukin 2 (IL-2), interleukin 10 (IL-10), and interferon gamma (IFN-γ) after an incubation of 6-48 h under gastro-intestinal conditions and selection of strains with an immunogenicity of not more than the negative control.

The gastric conditions for steps 1) and 7) may include incubation of strains at pH 1-4 for a time of between 30 minutes and 300 minutes at a temperature between 35° C. and 39° C. in simulated gastric fluid containing pepsin (0.5-6 g/l) and the intestinal conditions for step 1) include incubation of strains at pH 5.5-pH 8.5 for a time of between 30 minutes and 300 minutes at a temperature between 35° C. and 39° C. in simulated intestinal fluid containing pancreatin (0.02-0.6% w/v) and bile salts (0.05-0.6%).

The activities of peptidases aminopeptidase type N (PepN); PepI, PepO, Prolyl endopeptidyl peptidase (PEP), PepX, and PepQ peptide hydrolase in step 4) may be determined using strains at a density between 7.0 and 11.0 log CFU/ml in the form of viable cells or cytoplasmic extracts thereof with peptide substrates with amino acid sequences suitable for detection of aminopeptidase type N (PepN), PepI, PepO, Prolyl endopeptidyl peptidase (PEP), PepX, and PepQ peptide hydrolase activities.

The peptidase activities in step 6) may be determined by using viable cells or cytoplasmic extracts thereof in buffered media (pH 6.0-9.0) at 35-39° C. for 1-12 h and the strains with a degradation capacity of all four epitopes of more than 95%, preferably more than 98% are selected.

The simulated gastrointestinal conditions in step 8) may include incubation of the strains selected in step 7) at a density between 7.0 and 11.0 log CFU/ml, their cytoplasm and/or Bacillus proteases at pH 2-4 for a time of between 30 minutes and 300 minutes at a temperature between 36.5° C. and 37° C. in simulated gastric fluid containing pepsin (0.5-6 g/l) and incubation of the strains selected in step 7), their cytoplasm and/or Bacillus proteases at pH 7.0-pH 8.5 for a time of between 30 minutes and 48 hours at a temperature between 36.5° C. and 37° C. in simulated intestinal fluid containing pancreatin (0.02-0.6% w/v) and bile salts (0.05-0.6%).

In a preferred configuration, the bacterial strains are derived from one or more of the following sources: soil, cereals (wheat, ryes, barley), cereal processing, sourdough, feces from humans, pigs, dogs, cats, rats, or mice, gastrointestinal tract specimens from humans, pigs, dogs, cats, rats, or mice.

In an advantageous configuration, the bacterial strains are selected from one or more of the following genera: Lactobacillus, Bacillus, Pediococcus and Weissella.

The process according to the present invention yields consortia of probiotic strains that provide a technical solution for digestion of gluten based on the following considerations:
Gluten/gliadin/glutenin degradation during human digestion is not beneficial per se, because incomplete degradation can lead to the formation of toxic and/or immunogenic peptides
Concerns have been expressed on the safety of currently available means to trigger gluten degradation in vivo, as these may do so only partially and can thereby induce or worsen gluten toxicity
Any attempt to trigger gluten degradation in vivo needs to make sure that such degradation is complete and leads to safe degradation products
Given the diversity of gluten-inherent peptide sequences with immunogenic potential, a combination of peptide hydrolases from different microbes is required to ensure complete degradation of all peptides
Such combination is preferably provided by a consortium of probiotic microorganisms that are metabolically active and synergize with each other in relevant parts of the gastrointestinal tract (i.e. the stomach and duodenum) to promote a safe, rapid, and complete digestion of gluten proteins from relevant food matrices to non-toxic, non-immunogenic small peptides or amino acids
We conceived that such a synergism can be achieved by combining acid- and bile-resistant bacterial strains with suitable protein/peptide substrate specificities and found that combinations of certain Lactobacillus sp. and Bacillus sp., including their cytoplasm extracts, from specific ecological niches are particularly useful for that.

The means how the probiotic consortia that have been selected according to our process can bring a benefit to people in need thereof is as follows:
(i) Safe clearance of intentionally or accidentally ingested gluten as a cure or complementing therapy for CD, WA, and NCGS patients. The possibility to return to a conventional, gluten-containing diet.
(ii) Safe clearance of intentionally or accidentally ingested gluten as a cure or complementing therapy for people with non-specific intestinal or extra-intestinal symptoms that may result from ingested gluten. The possibility to return to a conventional, gluten-containing diet.
(iii) Offering a solution for symptom-free people wanting to minimize their gluten exposure as an alternative to adhere to a GFD.

### WORKING EXAMPLES

### Description of the Process Steps

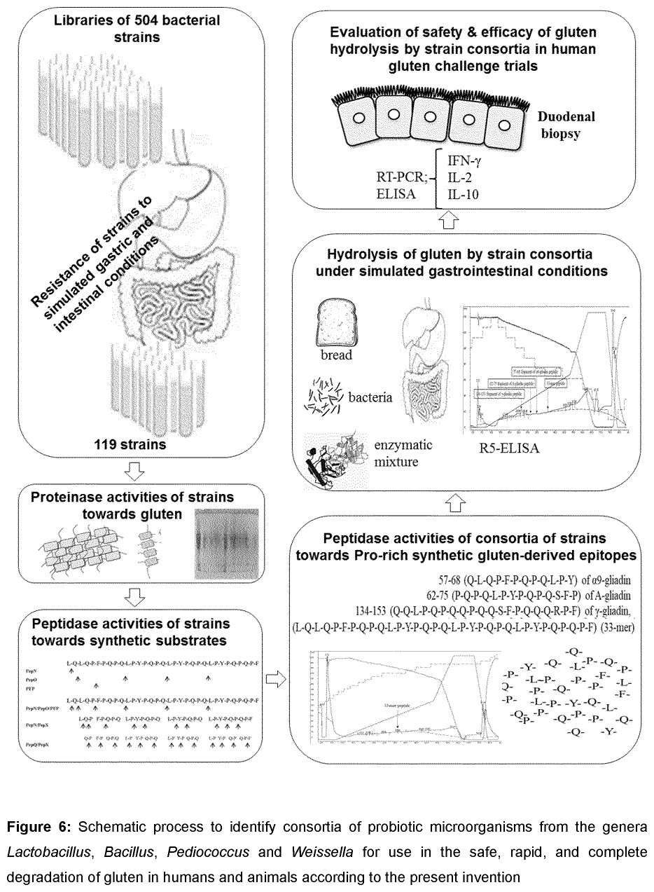

An overview of the process is shown in FIG. 6.

### Step 1: Compiling of Libraries of Bacterial Strains

The following libraries contain bacterial strains may be eligible as a starting point for the current invention. For example, four libraries of strains from the genera Lactobacillus (Library 1), Bacillus (Library 2), Pediococcus (Library 3) and Weissella (Library 4) are compiled. Each library contains at least ten different strains. The strains are derived from the following sources: soil, cereals (wheat, ryes, barley), cereal processing, sourdough, feces from humans, pigs, dogs, cats, rats, or mice, gastrointestinal tract specimens from humans, pigs, dogs, cats, rats, or mice. Strains belong to the following genera:

### Library 1=Lactobacillus sp.

Strains may belong to e.g. Lactobacillus plantarum (Lactiplantibacillus plantarum), Lactobacillus paracasei (Lacticaseibacillus paracasei), Lactobacillus sanfranciscensis (Fructilactobacillus sanfranciscensis), Lactobacillus brevis (Levilactobacillus brevis), Lactobacillus casei (Lacticaseibacillus casei), Lactobacillus rossiae, Lactobacillus fermentum, Lactobacillus acidophilus, Lactobacillus crispatus, Lactobacillus curvatus, Lactobacillus delbrueckii, Lactobacillus gasseri, Lactobacillus helveticus, Lactobacillus hilgardii, Lactobacillus johnsonii, Lactobacillus kefirii, Lactobacillus mucosae, Lactobacillus reuteri (Limosilactobacillus reuteri), Lactobacillus rhamnosus, Lactobacillus sakei, or Lactobacillus salivarius. Preferably, strains belong to the species Lactobacillus plantarum (Lactiplantibacillus plantarum), Lactobacillus paracasei (Lacticaseibacillus paracasei), Lactobacillus sanfranciscensis (Fructilactobacillus sanfranciscensis), Lactobacillus brevis (Levilactobacillus brevis), or Lactobacillus casei (Lacticaseibacillus casei).

### Library 2=Bacillus sp.

Strains may belong to e.g. Bacillus subtilis, Bacillus pumilus, Bacillus licheniformis, Bacillus amyloliquefaciens group, Bacillus coagulans, Bacillus fusiformis, or Bacillus megaterium. Preferably, strains belong to the species Bacillus subtilis, Bacillus pumilus, Bacillus licheniformis, or Bacillus megaterium.

### Library 3=Pediococcus sp.

Strains may belong to e.g. Pediococcus acidilactici, Pediococcus dextrinicus, Pediococcus parvulus, or Pediococcus pentosaceus.

### Library 4=Weissella sp.

Strains may belong to e.g. Weissella confusa, Weissella cibaria, Weissella halotolerans, Weissella kandleri, or Weissella paramesenteroides.

### Step 2: Resistance of Strains to Simulated Gastric and Intestinal Conditions

Simulated gastric and intestinal fluids were prepared and used as described by Fernandez et al. [19]. Stationary-phase-grown cells were harvested at 8000 g for 10 min, washed with physiologic solution, and suspended in 50 ml of simulated gastric juice (cell density of 10 log CFU/ml), which contains NaCl (125 mM/l), KCl (7 mM/l), NaHCO₃ (45 mM/l), and pepsin (3 g/l) [20]. The final pH was adjusted to 2.0, 3.0, and 8.0. The value of pH 8.0 was used to investigate the influence of the components of the simulated gastric juice, apart from the effect of low pH [19]. The suspension was incubated at 37° C. under anaerobic conditions and agitation to simulate peristalsis. Aliquots of this suspension were taken at 0, 90, and 180 min, and viable count was determined. The effect of gastric digestion was also determined by suspending cells in reconstituted skimmed milk (RSM) (11% solids, w/v) before inoculation of simulated gastric juice at pH 2.0. The final pH after the addition of RSM was ca. 3.0. This condition was assayed to simulate the effect of the food matrix during gastric transit [20]. After 180 min of gastric digestion, cells were harvested and suspended in simulated intestinal fluid, which contains 0.1% (w/v) pancreatin and 0.15% (w/v) Oxgall bile salt (at pH 8.0. The suspension was incubated at 37° C. under agitation and aliquots were taken at 0, 90, and 180 min [21].

Selection criterion=less than 2 log loss of CFU.

### Step 3: Proteinase Activities of Strains Towards Gluten

24-hour old cells of bacteria strains were harvested by centrifugation (12,400×g for 10 min at 4° C.), washed with sterile 0.05 M potassium phosphate buffer, pH 7.0, re-suspended in the same buffer at a 620 nm absorbance (A620) of 2.5, which corresponded to a cell density of ca. 9.0 log CFU/ml, and used for the enzyme assays. Proteinase (cell-envelope-associated proteinase) activity was measured using wheat flour proteins as substrates. Wheat flour proteins were separately extracted from wheat flour following the method of Weiss et al. [22]. The assay mixture, containing 4 mg/ml of albumins/globulins, gliadins or glutenins in 0.05 M potassium phosphate buffer, pH 7.0, and 0.1 ml of cellular suspension (ca. 9.0 log CFU/ml), was incubated at 37° C. for 180 min under stirring conditions (150 rpm). Sodium dodecyl sulfate-polyacrylamide gel electrophoresis (SDS-PAGE) was carried out on 12.5% acrylamide gels stained with B10 Bio-Safe Coomassie blue. Low-range SDS-PAGE molecular mass standards were used. Three gels for each assay were analyzed for protein band intensities by Quantity One software package. Gliadin (4 mg/ml) was suspended in gastric juice pH 2.0 and was incubated at 37° C. for 180 min under stirring conditions (150 rpm). After gastric digestion, hydrolyzed gliadin was centrifuged at 10,000 rpm for 10 min and then intestinal juice with (treated) or without (control) 0.1 ml of cellular suspension (ca. 9.0 log CFU/ml) was added to supernatant (which contains soluble peptides) and pellet (which contains insoluble peptides and proteins), separately. The assay mixture was incubated at 37° C. for 180 min under stirring conditions (150 rpm). An aliquot of intestinal suspension was incubated for 30 h at 37° C. under stirring conditions (150 rpm).

The protein concentration was determined by the Bradford method [23]. The concentration of peptides was determined by the o-phtaldialdehyde (OPA) method (Church F C, Swaisgood H E, Porter D H, Catignani G L. 1983. Spectrophotometric assay using o-phthaldialdehyde for determination of proteolysis in milk and isolated milk proteins. J. Dairy Sci. 66:1219-1227). A standard curve prepared using tryptone (0.25 to 1.5 mg/ml) was used as the reference. The use of peptone gave a similar standard curve. Immunological analysis was carried out by using R5 antibody-based sandwich and competitive ELISA (R5-ELISA). The R5-ELISA according to Valdes et al. [24] was carried out with the RIDASCREEN® Gliadin competitive detection kit according to the instructions of the manufacturer (R-Biopharm AG, Germany).

Selection criterion: very high gluten degradation compared to the other strains.

### Step 4: Peptidase Activities of Strains Towards Synthetic Substrates

To assay the cytoplasm peptidase activities, cultures of each strain from the late exponential phase of growth (ca. 9.0 log CFU/ml) were used. Aliquots (0.3 g [dry weight]) of washed cell pellets were re-suspended in 50 mM Tris-HCl (pH 7.0), incubated at 30° C. for 30 min, and centrifuged at 13,000×g for 10 min to remove enzymes loosely associated to cell surface. The cytoplasmic extract was prepared by incubating bacterial suspensions with lysozyme in 50 mM Tris-HCl (pH 7.5) buffer containing 24% sucrose at 37° C. for 60 min, under stirring conditions (ca. 160 rpm). Spheroplasts were resuspended in isotonic buffer and sonicated for 40 sat 16 A/s (Sony Prep model 150; Sanyo, United Kingdom). The cytoplasmic extract was concentrated 10-fold by freeze-drying, re-suspended in 5 mM Tris-HCl (pH 7.0), and dialyzed for 24 h at 4° C.

General aminopeptidase type N (PepN), proline iminopeptidase (PepI), X-prolyl dipeptidyl aminopeptidase (PepX) activities of the cytoplasmic extracts of lactobacilli were measured by using Leu-p-nitroanilides (p-NA), Pro-p-NA and Gly-Pro-p-NA substrates, respectively. The assay mixture contained 900 μl of 2.0 mM substrate in 0.05 M potassium phosphate buffer, pH 7.0, and 100 μl of cytoplasmic extract. The mixture was incubated at 37° C. for 180 min, and the absorbance was measured at 410 nm. The data were compared to standard curves set up by using p-nitroaniline. One unit of activity was defined as the amount of enzyme required to liberate 1 μmol of p-nitroaniline for min under the assay conditions.

Selection criterion=very high activity for at least one of the enzymes compared to the other strains.

### Step 6: Peptidase Activities of Consortia of Strains Towards Pro-Rich Synthetic Gluten-Derived Epitopes

Various mixtures of strains (consortium) were used to assay their capacity to in vitro degrade immunogenic epitopes responsible for the gluten intolerance. Immunogenic epitopes corresponding to fragments 57-68 (Q-L-Q-P-F-P-Q-P-Q-L-P-Y) of α9-gliadin, 62-75 (P-Q-P-Q-L-P-Y-P-Q-P-Q-S-F-P) of A-gliadin, 134-153 (Q-Q-L-P-Q-P-Q-Q-P-Q-Q-S-F-P-Q-Q-Q-R-P-F) of y-gliadin, and 57-89 (L-Q-L-Q-P-F-P-Q-P-Q-L-P-Y-P-Q-P-Q-L-P-Y-P-Q-P-Q-L-P-Y-P-Q-P-Q-P-F) (33-mer) of α2-gliadin were chemically synthesized. The hydrolysis of peptides was carried out using the cytoplasmic extract of previously selected bacteria strains. The mixture, containing 100 μl of cytoplasmic extract(s) and 2 mM synthetic peptide in 1 ml of 50 mM phosphate buffer (pH 7.5) was incubated for 180 min at 37° C. while being stirred (150 rpm). Hydrolysis of peptides was monitored searching for liberated peptides through HPLC analysis, respectively. Liquid chromatography coupled with electrospray ionization (ESI)-ion trap mass spectrometry (MS) was used to complete the analysis.

Selection criterion=degradation of all four epitopes.

### Step 7: Hydrolysis of Gluten by Strain Consortia Under Simulated Gastrointestinal Conditions

We used commercial gliadin for testing capacity of hydrolyzing gluten, related to the procedure described by Francavilla et al. [16]. Gliadin (4 mg/ml) was suspended in a simulated gastric juice that contain NaCl (125 mM/L), KCl (7 mM/L), NaHCO3 (45 mM/L), and pepsin (3 g/L). The final pH was adjusted to 2.0 with HCl. The suspension was incubated at 37° C. under anaerobic conditions and stirred to simulate peristalsis. After 180 min of gastric digestion, hydrolyzed gliadin was centrifuged at 10,000 rpm for 10 min and supernatant (which contains soluble peptides) and pellet (which contains insoluble peptides and proteins), were separately added with simulated intestinal fluid, which contained 0.1% (w/v) pancreatin and 0.15% (w/v) Oxgall bile salt (Sigma-Aldrich Co.) at pH 8.0. Simulated intestinal juice, with (treated) or without (control) 0.1 ml of cellular suspension (ca. 9.0 log CFU/ml) was incubated at 37° C. for 180 min under stirring conditions (150 rpm). The protein concentration was determined by the Bradford method [23]. The concentration of peptides was determined by the o-phtaldialdehyde (OPA) method (Church F C, Swaisgood H E, Porter D H, Catignani G L. 1983. Spectrophotometric assay using o-phthaldialdehyde for determination of proteolysis in milk and isolated milk proteins. J. Dairy Sci. 66:1219-1227). A standard curve prepared using tryptone (0.25 to 1.5 mg/ml) was used as the reference. The use of peptone gave a similar standard curve. Immunological analysis was carried out by using R5 antibody-based sandwich and competitive ELISA (R5-ELISA). The R5-ELISA [24] was carried out with the RIDASCREEN® Gliadin competitive detection kit according to the instructions of the manufacturer (R-Biopharm AG, Germany).

Selection criterion: relevant hydrolysis of gluten during incubation.

Step 9: Evaluation of Safety of Gluten Hydrolysis by Strain Consortia Using Small Intestinal Tissue Explants from CD Patients

Duodenal biopsy specimens were obtained from 10 CD patients (age range, 19 to 30 years) following a GFD. All CD patients expressed the HLA-DQ2 phenotype. CD was diagnosed according to European Society for Pediatric Gastroenterology, Hepatology, and Nutrition criteria (European Society of Paediatric Gastroenterology and Nutrition. 1990. Revised criteria for diagnosis of coeliac disease. Report of working group of European Society of Paediatric Gastroenterology and Nutrition. Arch Dis Child 65:909-911). Immediately after excision, all biopsy specimens were placed in ice-chilled culture medium (RPMI 1640; Gibco-Invitrogen, UK) and transported to the laboratory within 30 min. Duodenal biopsy specimens were cultured for 4 h using the organ tissue culture method originally described by Browning and Trier [25]. Briefly, the biopsy specimens were oriented villous side up on a stainless steel mesh and positioned over the central well of an organ tissue culture dish (Falcon, USA). The well contained RPMI supplemented with 15% fetal calf serum (Gibco-Invitrogen) and 1% penicillin-streptomycin. The dishes were placed in an anaerobic jar and incubated at 37° C.

Four biopsy specimens from each CD patient were cultured with culture medium under four conditions: (i) with doughs containing a mixture of bacterial strains and the enzymatic mixture (E1, E2, Veron PS, Veron HPP) digested for 48 h; (ii) with dough containing a different mixture of bacterial strains and enzymatic mixture (E1, E2, Veron PS, Veron HPP) digested for 48 h; (iii) with control dough digested for 48 h (Control); and (iv) with culture medium (RPMI 1640+gastric and intestinal juice, negative control).

Biopsy specimens from each patient were rinsed and stored in RNA later at −80° C. to preserve the RNA. Total RNA was extracted from the tissues using the RNeasy minikit (Qiagen GmbH) according to the manufacturer's instructions. The concentration of mRNA was estimated by determination of the UV absorbance at 260 nm. Aliquots of total RNA (500 ng) were reverse transcribed using random hexamers, TaqMan reverse transcription reagents, and 3.125 U/μl of MultiScribe reverse transcriptase to a final volume of 50 μl. The cDNA samples were stored at −20° C.

RT-PCR for IFN-γ, IL-2, and IL-10 genes: RT-PCR was performed in 96-well plates using an ABI Prism 7500HT fast sequence detection system (Applied Biosystems). Data collection and analyses were performed using the machine software. PCR primers and fluorogenic probes for the target genes (IFN-γ, IL-2, and IL-10) and the endogenous control (gene coding for glyceraldehyde-3-phosphate dehydrogenase [GAPDH]) were purchased as a TaqMan gene expression assay and a pre-developed TaqMan assay, respectively. The assays were supplied as a 20× mix of PCR primers and TaqMan Minor Groove Binder 6-carboxyfluorescein dye-labeled probes with a non-fluorescent quencher at the 3′ end of the probe. Two-step reverse transcription-PCR was performed using first-strand cDNA with a final concentration of 1×TaqMan gene expression assay mix and 1×TaqMan universal PCR master mix. The final reaction volume was 25 μl. Each sample was analyzed in triplicate, and all experiments were repeated twice. A non-template control (RNase-free water) was included with every plate. The following thermal cycler conditions were used: 2 min at 50° C. (uracil DNA glycosylase activation), 10 min at 95° C., and 40 cycles of 15 s at 95° C. and 1 min at 60° C. Initially, a standard curve and a validation experiment were performed for each primer/probe set. Six serial dilutions (20 to 0.1 ng/μl) of IFN-γ, IL-2, or IL-10 cDNA were used as a template for each primer/probe set. A standard curve was generated by plotting the threshold cycle (CT) values against the log of the amount of input cDNA. The CT value is the PCR cycle at which an increase in reporter fluorescence above the baseline level is first detected. The average value for the target gene was normalized using an endogenous reference gene (the GAPDH gene). A healthy duodenal biopsy specimen was used to calibrate all the experiments. The levels of IFN-γ, IL-2, and IL-10 proteins secreted into the supernatant were quantified by ELISA in 96-well round-bottom plates (Tema Ricerca, Milan, Italy) according to the manufacturer's recommendations.

Selection criterion=non-immunogenicity of the digest.

### Example 1. Probiotic Microorganisms Resistant to Gastrointestinal Conditions

Simulated gastric and intestinal fluids were used as described by Fernandez et al. [19]. Stationary-phase-grown cells were harvested at 8000 g for 10 min, washed with physiologic solution, and suspended in 50 ml of simulated gastric juice (cell density of 10 log CFU/ml), which contains NaCl (125 mM/l), KCl (7 mM/l), NaHCO₃ (45 mM/l), and pepsin (3 g/l) (Sigma-Aldrich CO., St. Louis, Mo., USA) [20]. The final pH was adjusted to 2.0, 3.0, and 8.0. The value of pH 8.0 was used to investigate the influence of the components of the simulated gastric juice, apart from the effect of low pH. The suspension was incubated at 37° C. under anaerobic conditions and agitation to simulate peristalsis. Aliquots of this suspension were taken at 0, 90, and 180 min, and viable count was determined. The effect of gastric digestion was also determined by suspending cells in reconstituted skimmed milk (RSM) (11% solids, w/v) before inoculation of simulated gastric juice at pH 2.0. The final pH after the addition of RSM was ca. 3.0. This condition was assayed to simulate the effect of the food matrix during gastric transit [20]. After 180 min of gastric digestion, cells were harvested and suspended in simulated intestinal fluid, which contains 0.1% (w/v) pancreatin and 0.15% (w/v) Oxgall bile salt (Sigma-Aldrich Co.) at pH 8.0. The suspension was incubated at 37° C. under agitation and aliquots were taken at 0, 90, and 180 min [21]. 119 out of <400 tested strains showed a decrease of less than 2 log of initial 1×10¹⁰ CFU/ml and defined as resistant to simulated gastrointestinal conditions.

### Example 2. Protease and Peptidase Activities of Single Strains Resistant to Gastrointestinal Conditions

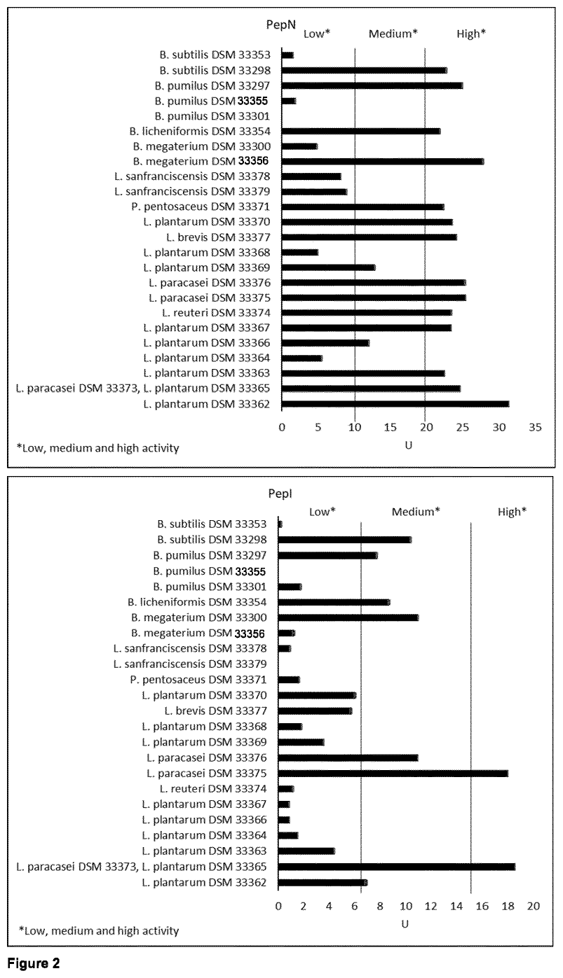

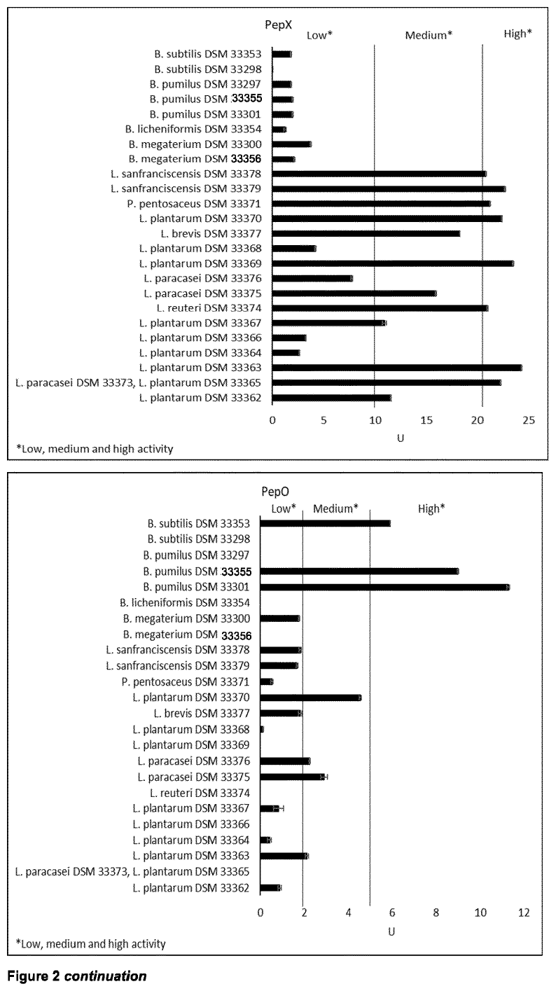

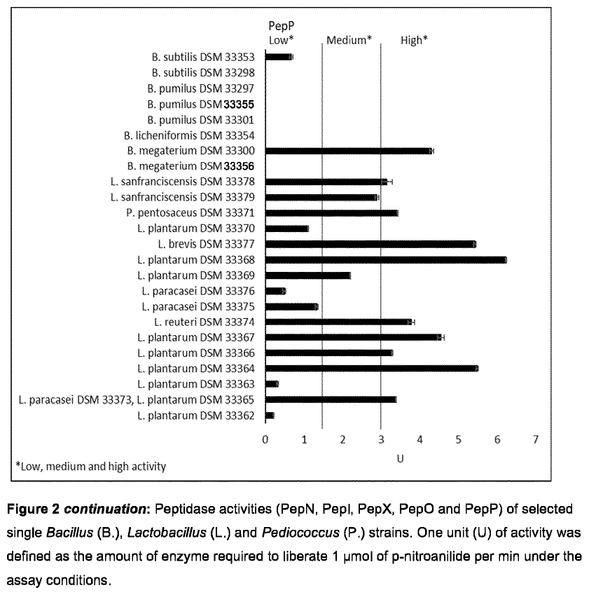

All 119 strains (Lactobacillus sp., 63 strains; Weissella sp., 3 strains; Pediococcus sp., 1 strain; and Bacillus sp., 51 strains) showing resistance to simulated gastrointestinal conditions were tested for their peptidase and proteinase activities towards synthetic substrates. To assay the peptidase activities, cultures of each strain from the late exponential phase of growth (ca. 9.0 log CFU/ml) were used. Aliquots (0.3 g [dry weight]) of washed cell pellets were re-suspended in 50 mM Tris-HCl (pH 7.0), incubated at 30° C. for 30 min, and centrifuged at 13,000×g for 10 min to remove enzymes loosely associated to the cell wall. The cytoplasmic extract was prepared by incubating bacterial suspensions with lysozyme in 50 mM Tris-HCl (pH 7.5) buffer containing 24% sucrose at 37° C. for 60 min, under stirring conditions (ca. 160 rpm). Spheroblasts were resuspended in isotonic buffer and sonicated for 40 s at 16 A/s (Sony Prep model 150; Sanyo, United Kingdom). The extracts were concentrated 10-fold by freeze-drying, re-suspended in 5 mM Tris-HCl (pH 7.0), and dialyzed for 24 h at 4° C. General aminopeptidase type N (PepN), proline iminopeptidase (PepI), X-prolyl dipeptidyl aminopeptidase (PepX) endopeptidase (PepO) and prolyl endopeptidase (PepP) activities of the cytoplasmic extracts of lactobacilli were measured by using Leu-p-nitroanilides (p-NA), Pro-p-NA, Gly-Pro-p-NA, Z-Gly-Gly-Leu-p-NA and Z-Gly-Pro-4-nitroanilide substrates (Sigma Chemical Co), respectively. The assay mixture contained 900 μl of 2.0 mM substrate in 0.05 M potassium phosphate buffer, pH 7.0, and 100 μl of cytoplasmic extract. The mixture was incubated at 37° C. for 180 min, and the absorbance was measured at 410 nm. The data were compared to standard curves set up by using p-nitroaniline. One unit of activity was defined as the amount of enzyme required to liberate 1 μmol of p-nitroaniline for min under the assay conditions. Based on Principal Component Analysis (PCA) data from the above peptidase activities, some strains clearly separated from the other ones (FIG. 1). FIG. 2 reports the strains showing very high peptidase activities (at least for one peptidase activity). PepN activity ranged from 0.0 (U002-C04; U541-C05; U776-C02; DSM 33301; U021-C01; DSM32540; U567-C04) to 31.400±0.09 U (DSM 33362) (median value 3.08). The strains with low peptidase activity (with the internal numbers/or deposited at the DSMZ: U002-C04; U541-C05; U776-C02; DSM 33301; U021-C01; DSM32540; U567-C04) were not further evaluated. The other most active strains were DSM 33367, DSM 33374, DSM 33370, DSM 33371, DSM 33377, DSM 33373, Bacillus pumilus DSM 33297, Bacillus subtilis DSM 33298, DSM 33376, DSM 33375, DSM 33363, Bacillus licheniformis DSM 33354, and Bacillus megaterium DSM 33356 (FIG. 1, FIG. 2). The median value of PepI was of 1.66. The most active strains (PepI activity >18 U) were DSM 33375, DSM 33373. PepX activity ranged from 0.0 to ca. 24 U. The most active strains were DSM 33379, DSM 33371, DSM 33370, DSM 33369, DSM 33374, DSM 33373, and DSM 33363 (FIG. 1 and FIG. 2) (median value of 1.81). The median value of PepO was of 0.54. The most active strains (PepO activity >5 U) were DSM 33353, DSM 33355, and DSM 33301. PepP activity ranged from 0.0 to 6.23 U (DSM 33368) (median value 0.22). The other most active strains (PepP activity >3 U) were Bacillus megaterium DSM 33300, DSM 33378, DSM 33371, DSM 33377, DSM 33367, DSM 33374, DSM 33366, DSM 33373, and DSM 33364.

FIG. 1 shows the score (A) and loading (B) plots of the first and second principal components after principal component analysis (PCA) based on the general aminopeptidase type N (PepN), proline iminopeptidase (PepI), X-prolyl dipeptidyl aminopeptidase (PepX), endopeptidase (PepO) and prolyl endopeptidase (PepP) activities of the cytoplasmic extracts of the 119 Bacillus, Lactobacillus, Pediococcus, and Weissella strains. PepN, PepI, PepX, PepP were measured by using Leu-p-nitroanilides (p-NA), Pro-p-NA, Gly-Pro-p-NA, Z-Gly-Gly-Leu-p-NA and Z-Gly-Pro-4-nitroanilide substrates, respectively. Strains showing very high peptidase activities (at least for one peptidase) were reported in red.

FIG. 2 shows peptidase activities (PepN, PepI, PepX, PepO and PepP) of selected single Bacillus (B.), Lactobacillus (L.) and Pediococcus (P.) strains. One unit (U) of activity was defined as the amount of enzyme required to liberate 1 μmol of p-nitroanilide per min under the assay conditions.

### Example 3. Peptidase Activities of Mixture of Strains Against Immunogenic Epitopes

Bacillus, Lactobacillus, and Pediococcus strains showing very high peptidase activities (at least for one peptidase) were assessed as mixed strains to combine intense and complementary enzyme activities. Various mixtures were used to assay their capacity to in vitro degrade immunogenic epitopes responsible for gluten intolerance.

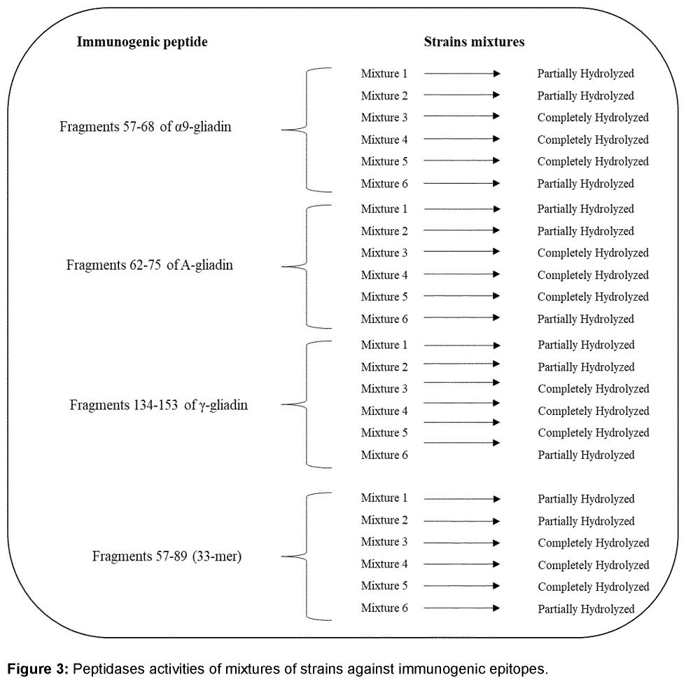

The hydrolysis of peptides was carried out using combinations of cytoplasmic extracts of previously selected bacteria strains. Immunogenic epitopes corresponding to fragments 57-68 (Q-L-Q-P-F-P-Q-P-Q-L-P-Y) of a9-gliadin, 62-75 (P-Q-P-Q-L-P-Y-P-Q-P-Q-S-F-P) of A-gliadin, 134-153 (Q-Q-L-P-Q-P-Q-Q-P-Q-Q-S-F-P-Q-Q-Q-R-P-F) of y-gliadin, and 57-89 (L-Q-L-Q-P-F-P-Q-P-Q-L-P-Y-P-Q-P-Q-L-P-Y-P-Q-P-Q-L-P-Y-P-Q-P-Q-P-F) (33-mer) of a2-gliadin were chemically synthesized and used at an initial concentration of 1 mM. Hydrolysis was monitored by RP-HPLC. Single peaks from RP-HPLC were analysed by nano-ESI tandem mass spectrometry (nano-ESI-MS/MS). The mixtures of strains that showed the best hydrolysis of synthetic immunogenic epitopes were numbers 3, 4 and 5 (FIG. 3), which fully hydrolysed all toxic peptides (90% hydrolysis or more). FIG. 3 shows peptidase activities of mixtures of strains against immunogenic epitopes.

Strain mixtures were as follows:
1. L. plantarum (Lactiplantibacillus plantarum) DSM 33362, DSM 33363, DSM 33364, DSM 33366; L. sanfranciscensis (Fructilactobacillus sanfranciscensis) DSM 33379; Bacillus pumilus DSM 33297, DSM 33355, Bacillus licheniformis DSM 33354, Bacillus megaterium DSM 33300, Bacillus subtilis DSM 33353.
2. L. paracasei (Lacticaseibacillus paracasei) DSM 33375, DSM 33376; L. plantarum (Lactiplantibacillus plantarum) DSM 33369, DSM 33368; L. sanfranciscensis (Fructilactobacillus sanfranciscensis) DSM 33378; Bacillus licheniformis DSM 33354, Bacillus megaterium DSM 33300, DSM 33356, Bacillus pumilus DSM 33297, DSM 33301.
3. L. plantarum (Lactiplantibacillus plantarum) DSM 33370, DSM 33363, DSM 33364; Lactobacillus paracasei (Lacticaseibacillus paracasei) DSM 33373, L. brevis (Levilactobacillus brevis) DSM 33377; Bacillus pumilus DSM 33297, DSM 33355, Bacillus licheniformis DSM 33354, Bacillus megaterium DSM 33300, Bacillus subtilis DSM 33353.
4. L. plantarum (Lactiplantibacillus plantarum) DSM 33362, DSM 33367, DSM 33368; L. paracasei (Lacticaseibacillus paracasei) DSM 33375; L. sanfranciscensis (Fructilactobacillus sanfranciscensis) DSM 33379; Bacillus pumilus DSM 33301, Bacillus megaterium DSM 33300, DSM 33356, and Bacillus subtilis DSM 33298, DSM 33353.
5. L. plantarum (Lactiplantibacillus plantarum) DSM 33366, DSM 33369, Lactobacillus reuteri (Limosilactobacillus reuteri) DSM 33374; L. paracasei (Lacticaseibacillus paracasei) DSM 33376; Pediococcus pentosaceus DSM 33371, L. sanfranciscensis (Fructilactobacillus sanfranciscensis) DSM 33378; Bacillus licheniformis DSM 33354, Bacillus pumilus DSM 33301, Bacillus megaterium DSM 33300, DSM 33356, Bacillus subtilis DSM 33298.
6. L. plantarum (Lactiplantibacillus plantarum) DSM 33370, DSM 33367, Lactobacillus reuteri (Limosilactobacillus reuteri) DSM 33374; L. brevis (Levilactobacillus brevis) DSM 33377; Bacillus pumilus DSM 33301, Bacillus megaterium DSM 33300, DSM 33356, Bacillus subtilis DSM 33298.

### Example 4. Degradation of Gluten Under Simulated Gastrointestinal Conditions by Different Consortia

The gluten degradation under simulated gastrointestinal digestion was assessed. With the intention to develop a feasible technical solution for full degradation of gluten in vivo, we searched for minimal combinations containing as few strains as possible and as many as needed.

Using mixtures 1-6 of Example 3 as a starting point, the following consortia, selected from a total of 22 strains (Lactobacillus plantarum (Lactiplantibacillus plantarum) DSM 33370, DSM 33362, DSM 33363, DSM 33364, DSM 33366, DSM 33368, DSM 33369 and DSM 33367; Lactobacillus reuteri (Limosilactobacillus reuteri) DSM 33374; Lactobacillus paracasei (Lacticaseibacillus paracasei) DSM 33376, Lactobacillus paracasei (Lacticaseibacillus paracasei) DSM 33373, DSM 33375; Lactobacillus brevis (Levilactobacillus brevis) DSM 33377, Pediococcus pentosaceus DSM 33371; Bacillus pumilus DSM 33297, DSM 33355, DSM 33301, DSM 33355, Bacillus licheniformis DSM 33354, Bacillus megaterium DSM 33300, DSM 33356, and Bacillus subtilis DSM 33298, DSM 33353) were prepared:
1. L. plantarum (Lactiplantibacillus plantarum) DSM 33370, DSM 33363 and DSM 33364, L. paracasei (Lacticaseibacillus paracasei) DSM 33373 L. brevis (Levilactobacillus brevis) DSM 33377, Bacillus pumilus DSM 33297, DSM 33355, DSM 33301;
2. L. plantarum (Lactiplantibacillus plantarum) DSM 33362 and DSM 33367, DSM 33368, L. paracasei (Lacticaseibacillus paracasei) DSM 33375, Bacillus subtilis DSM 33298, Bacillus licheniformis DSM 33354, and Bacillus megaterium DSM 33300;
3. L. plantarum (Lactiplantibacillus plantarum) DSM 33366, DSM 33369, Lactobacillus reuteri (Limosilactobacillus reuteri) DSM 33374, L. paracasei (Lacticaseibacillus paracasei) DSM 33376, Pediococcus pentosaceus DSM 33371, Bacillus megaterium DSM 33356, and Bacillus subtilis DSM 33353;
4. L. plantarum (Lactiplantibacillus plantarum) DSM 33363 and DSM 33364, L. paracasei (Lacticaseibacillus paracasei) DSM 33373, Bacillus subtilis DSM 33298 and Bacillus pumilus DSM 33301;
5. L. brevis (Levilactobacillus brevis) DSM 33377, Pediococcus pentosaceus DSM 33371, L. plantarum (Lactiplantibacillus plantarum) DSM 33369, Bacillus pumilus DSM 33297 and Bacillus megaterium DSM 33300;
6. L. paracasei (Lacticaseibacillus paracasei) DSM 33375, L. plantarum (Lactiplantibacillus plantarum) DSM 33367, DSM 33368; Bacillus pumilus DSM 33355, and Bacillus licheniformis DSM 33354;
7. L. plantarum (Lactiplantibacillus plantarum) DSM 33370, DSM 33362, and DSM 33366, Lactobacillus reuteri (Limosilactobacillus reuteri) DSM 33374, Bacillus megaterium DSM 33356, and Bacillus subtilis DSM 33353.
8. L. plantarum (Lactiplantibacillus plantarum) DSM 33363, DSM 33364, L. paracasei (Lacticaseibacillus paracasei) DSM 33375, L. reuteri (Limosilactobacillus reuteri) DSM 33374, B. megaterium DSM 33300, B. pumilus DSM 33297;
9. L. paracasei (Lacticaseibacillus paracasei) DSM 33375, L. plantarum (Lactiplantibacillus plantarum) DSM 33367, L. reuteri (Limosilactobacillus reuteri) DSM 33374, B. megaterium DSM 33300, B. pumilus DSM 33297, B. licheniformis DSM 33354;
10. L. plantarum (Lactiplantibacillus plantarum) DSM 33363, DSM 33364, DSM 33370, L. brevis (Levilactobacillus brevis) DSM 33377, B. pumilus DSM 33297, Bacillus megaterium DSM 33356;
11. L plantarum (Lactiplantibacillus plantarum) DSM 33362, DSM 33367, DSM 33368, L. paracasei (Lacticaseibacillus paracasei) DSM 33375, B. megaterium DSM 33300, B. subtilis DSM 33353;
12. L. plantarum (Lactiplantibacillus plantarum) DSM 33366, DSM 33369, L. reuteri (Limosilactobacillus reuteri) DSM 33374, L. paracasei (Lacticaseibacillus paracasei) DSM 33376, P. pentosaceus DSM 33371, B. pumilus DSM 33297, DSM 33355;
13. L. brevis (Levilactobacillus brevis) DSM 33377, P. pentosaceus DSM 33371, L. sanfranciscensis (Fructilactobacillus sanfranciscensis) DSM 33379, B. megaterium DSM 33300, B. pumilus DSM 33297;
14. L. plantarum (Lactiplantibacillus plantarum) DSM 33368, L. paracasei (Lacticaseibacillus paracasei) DSM 33375, L. sanfranciscensis (Fructilactobacillus sanfranciscensis) DSM 33378, B. megaterium DSM 33300, B. pumilus DSM 33297, B. licheniformis DSM 33354;
15. L. plantarum (Lactiplantibacillus plantarum) DSM 33362, DSM 33366, DSM 33370, L. reuteri (Limosilactobacillus reuteri) DSM 33374, L. sanfranciscensis (Fructilactobacillus sanfranciscensis) DSM 33378, DSM 33379, B. licheniformis DSM 33354, B. subtilis DSM 33353;
16. L. plantarum (Lactiplantibacillus plantarum) DSM 33363, DSM 33364, L. paracasei (Lacticaseibacillus paracasei) DSM 33373, L. reuteri (Limosilactobacillus reuteri) DSM 33374, B. megaterium DSM 33300, B. pumilus DSM 33297, DSM 33355.

Five grams of wheat bread (chewed for 30 s and collected in a beaker with 10 mL of NaK-phosphate 0.05 M, pH 6.9) or related dough were suspended in simulated gastric juice containing NaCl (125 mM), KCl (7 mM), NaHCO₃ (45 mM), and pepsin (3 g/L) (Sigma-Aldrich CO., St. Louis, Mo., USA). The suspension was added of the pooled selected strains as live (with a final cell density of approximately 9.0 log CFU/mL) and lysed bacteria (corresponding to 9.0 log cells/mL). The calculated initial amount of gluten in the reaction mixture was 7.000 ppm. A control dough, without addition of bacterial mixture, was also subjected to simulated digestion. The suspension was incubated at 37° C., under stirring to simulate peristalsis. After 180 min of gastric digestion, the suspension was added with simulated intestinal fluid, which contained 0.1% (w/v) pancreatin and 0.15% (w/v) Oxgall bile salt (Sigma-Aldrich Co.) at pH 8.0. Besides pancreatin and bile salt, the fluid contained enzymatic preparation E1, E2 (each at 0.2 g/kg), Veron HPP (10 g/100 kg of protein) and Veron PS (25 g/100 kg of protein) enzymes. Proteases of Aspergillus oryzae (500,000 haemoglobin units on the tyrosine basis/g; enzyme 1 [E1]) and Aspergillus niger (3,000 spectrophotometric acid protease units/g; enzyme 2 [E2]), routinely used for bakery applications, were supplied by BIO-CAT Inc. (Troy, Va.). Veron HPP and Veron PS are bacterial proteases from Bacillus subtilis (AB Enzymes). Enzymatic mixture (E1, E2, Veron PS, Veron HPP) was not added in the control dough. Intestinal digestion was carried out for 48 h at 37° C. under stirring conditions (ca. 200 rpm). After digestion, samples were put on ice and the concentration of hydrolysed gluten was determined according to a AOAC (Association of Official Agricultural Chemists) International Official Method of Analysis (OMA) (Method No. AACCI 38-55.01) using R5 antibody-based sandwich and competitive ELISA (R5-ELISA) [22]. R5-ELISA analysis was carried out with the RIDASCREEN® Gliadin competitive detection kit according to the instructions of the manufacturer (R-Biopharm AG, Germany). Moreover, ELISA Systems Gluten Residue Detection Kit (Windsor, Australia) was used for quantification of residual gluten. The presence of epitopes in digested samples was monitored after 6, 16, 24, 36 and 48 h of incubation through HPLC analysis. Liquid chromatography coupled with nano electrospray ionization-ion trap tandem mass spectrometry (nano-ESI-MS/MS) was also used to confirm the hydrolysis of gluten and the absence of toxic epitopes.

As estimated by the R5-ELISA (AOAC Official Method of Analysis, Method No. AACCI 38-55.01), after 6 h of digestion the concentration of hydrolysed gluten was in the range of 810±0.02 ppm for the control and 310±0.06 ppm for mixture 3 (Table 2). After 16 and 24 h of digestion, gluten content was 100 ppm for most of the mixtures. Importantly, gluten fragment levels were below 20 ppm after 36 h of digestion with mixtures 4 and 16; while gluten fragments were completely absent at the end of incubation (48 h) for mixture 4, 5, 6, 8, and 16.

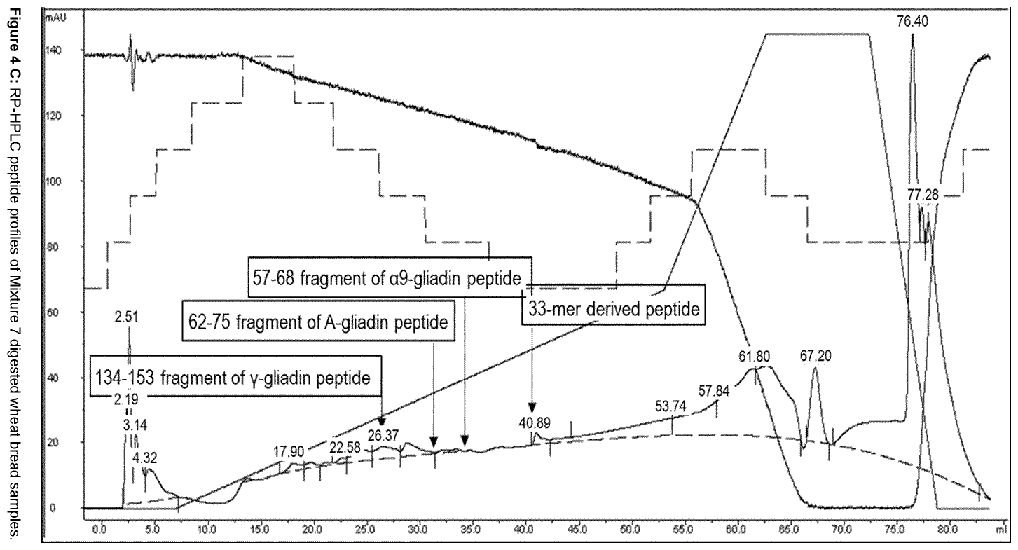

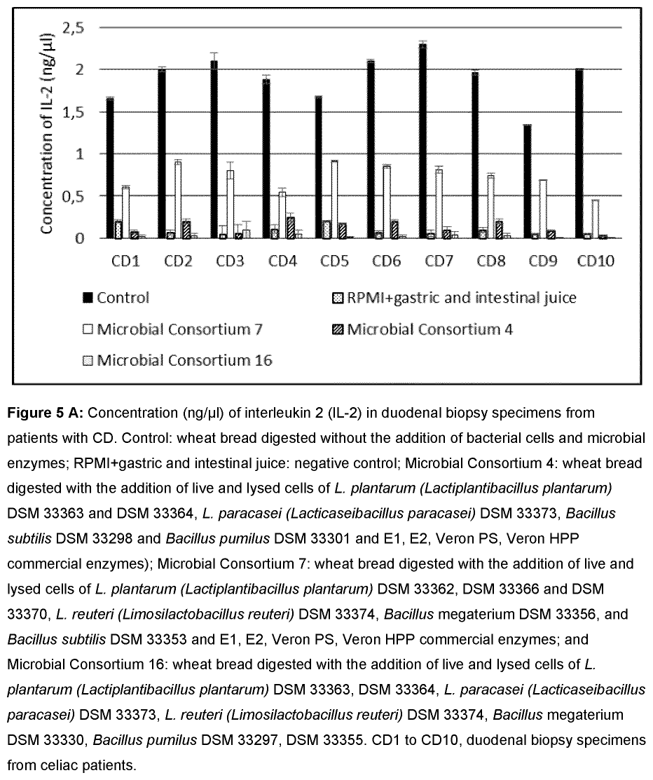

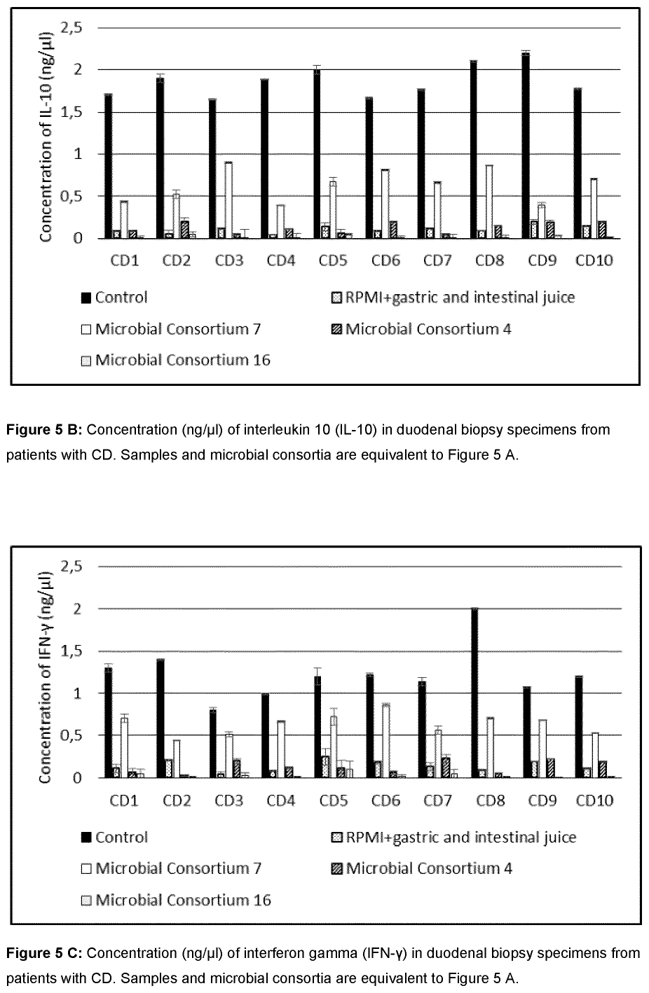

Regarding the residual gluten, most of the mixtures (MC1-9, 16) reduced it below the critical threshold of 20 ppm within 24 h of digestion. Furthermore, mixtures 4-9 and 16 were able to decrease residual gluten to ≤20 ppm within 16 h. Most importantly, the mixture 4 showed complete after 16 h of digestion (FIG. 5). MC8 and MC16 resulted in complete gluten degradation already within the first six hours of digestion. In total, MC4, MC8, and MC16 caused the most efficient removal of intact as well as fragmented gluten (Table 2).

TABLE 2
Concentration (ppm) of residual gluten and peptide fragments of prolamins after 6, 16, 24, 36, and 48 h of simulated
gastrointestinal digestion, as estimated by specific ELISA tests. Control: dough digested without bacterial cells
and commercial enzymes; MC1-MC16: microbial consortia constructed by using live and lysed cells of selected Lactobacillus
(L.) and Bacillus (B.) strains and E1, E2, Veron PS, and Veron HPP commercial enzymes.

| | | | | | | | Competitive |
|---|---|---|---|---|---|---|---|
| | | | | | | | ELISA Assay |
| | | | | | | | (Peptide |
| | | Sandwich ELISA Assay (Residual Gluten) | | | | | Fragments) |
| Strains | | 6 h | 16 h | 24 h | 36 h | 48 h | 6 h |
| Control | | 1100^{a} ± 0.06 | 620^{a} ± 0.09 | 367^{a} ± 0.05 | 256^{a} ± 0.04 | 75^{a} ± 0.06 | 810^{a} ± 0.03 |
| MC1 | Lp. plantarum DSM33370, DSM33363, | 406^{b} ± 0.04 | 135^{b} ± 0.06 | 19^{e} ± 0.01 | 0^{e} | 0^{e} | 310^{f} ± 0.05 |
| | DSM33364; Lc. paracasei DSM33373; | | | | | | |
| | Lv. brevis DSM33377; B. pumilus | | | | | | |
| | DSM33297, DSM33355, DSM33301 | | | | | | |
| MC2 | Lp. plantarum DSM33362, DSM33367, | 346^{c} ± 0.07 | 121^{b} ± 0.03 | 15^{e} ± 0.01 | 0^{e} | 0^{e} | 332^{f} ± 0.05 |
| | DSM33368; L. paracasei DSM33375; | | | | | | |
| | B. subtilis DSM33298; B. licheniformis | | | | | | |
| | DSM33354; B. megaterium DSM33300 | | | | | | |
| MC3 | Lp. plantarum DSM33366, DSM33369; | 382^{c} ± 0.03 | 99^{c} ± 0.02 | 12^{f} ± 0.01 | 0^{e} | 0^{e} | 315^{f} ± 0.06 |
| | Ls. reuteri DSM33374; Lc. paracasei | | | | | | |
| | DSM33376; Ped. pentosaceus DSM33371; | | | | | | |
| | B. megaterium DSM33356; B. subtilis | | | | | | |
| | DSM33353 | | | | | | |
| MC4 | Lp. plantarum DSM33363, DSM33364; | 190^{cd} ± 0.05 | 0^{g} | 0^{g} | 0^{e} | 0^{e} | 399^{e} ± 0.08 |
| | Lc. paracasei DSM33373; B. subtilis | | | | | | |
| | DSM33298; B. pumilus DSM33301 | | | | | | |
| MC5 | Lv. brevis DSM33377; Ped. | 380^{b} ± 0.06 | 18^{e} ± 0.01 | 5^{g} ± 0.01 | 0^{e} | 0^{e} | 398^{e} ± 0.04 |
| | pentosaceus DSM33371; Lp. plantarum | | | | | | |
| | DSM33369; B. pumilus DSM33297; | | | | | | |
| | B. megaterium DSM33300 | | | | | | |
| MC6 | Lc. paracasei DSM33375; Lp. | 350^{c} ± 0.06 | 15^{e} ± 0.02 | 2^{g} ± 0.01 | 0^{e} | 0^{e} | 404^{e} ± 0.06 |
| | plantarum DSM33367, DSM33368; | | | | | | |
| | B. pumilus DSM33355; B. licheniformis | | | | | | |
| | DSM33354 | | | | | | |
| MC7 | Lp. plantarum DSM33370, DSM33362, | 360^{c} ± 0.09 | 20^{e} ± 0.06 | 10^{f} ± 0.01 | 0^{e} | 0^{e} | 401^{e} ± 0.07 |
| | DSM33366; Ls. reuteri DSM33374; | | | | | | |
| | B. megaterium DSM33356; B. subtilis | | | | | | |
| | DSM33353 | | | | | | |
| MC8 | Lp. plantarum DSM33363, DSM33364; | 18^{g} ± 0.03 | 3^{g} ± 0.01 | 0^{g} | 0^{e} | 0^{e} | 323^{f} ± 0.08 |
| | Lc. paracasei DSM33375; Ls. reuteri | | | | | | |
| | DSM33374; B. megaterium DSM33300; | | | | | | |
| | B. pumilus DSM33297 | | | | | | |
| MC9 | Lc. paracasei DSM33375; Lp. plantarum | 60^{f} ± 0.04 | 12^{f} ± 0.01 | 0^{g} | 0^{e} | 0^{e} | 319^{f} ± 0.06 |
| | DSM33367; Ls. reuteri DSM33374; | | | | | | |
| | B. megaterium DSM33300; B. pumilus | | | | | | |
| | DSM33297; B. licheniformis DSM33354 | | | | | | |
| MC10 | Lp. plantarum DSM33363, DSM33364, | 112^{e} ± 0.06 | 77^{d} ± 0.04 | 70^{d} ± 0.02 | 0^{e} | 0^{e} | 465^{d} ± 0.09 |
| | DSM33370; Lv. brevis DSM33377; | | | | | | |
| | B. pumilus DSM33297; B. megaterium | | | | | | |
| | DSM33356 | | | | | | |
| MC11 | Lp. plantarum DSM33368, DSM33362, | 221^{d} ± 0.05 | 89^{c} ± 0.07 | 69^{d} ± 0.06 | 50^{d} ± 0.04 | 43^{d} ± 0.03 | 512^{c} ± 0.06 |
| | DSM33367; Lc. paracasei DSM33375; | | | | | | |
| | B. megaterium DSM33300; B. subtilis | | | | | | |
| | DSM33353 | | | | | | |
| MC12 | Lp. plantarum DSM33366, DSM33369; | 145^{e} ± 0.06 | 110^{c} ± 0.05 | 89^{c} ± 0.03 | 75^{c} ± 0.02 | 63^{b} ± 0.03 | 601^{b} ± 0.09 |
| | Ls. reuteri DSM33374; Lc. paracasei | | | | | | |
| | DSM33376; Ped. pentosaceus | | | | | | |
| | DSM33371; B. pumilus DSM33297, | | | | | | |
| | DSM33355 | | | | | | |
| MC13 | Lv. brevis DSM33377; Ped. | 163^{de} ± 0.06 | 122^{b} ± 0.04 | 82^{c} ± 0.02 | 45^{d} ± 0.03 | 0^{e} | 523^{c} ± 0.07 |
| | pentosaceus DSM33371; Fr. | | | | | | |
| | sanfranciscensis DSM33379; B. | | | | | | |
| | megaterium DSM33300; B. pumilus | | | | | | |
| | DSM33297 | | | | | | |
| MC14 | Lp. plantarum DSM33368; Lc. | 234^{d} ± 0.08 | 135^{b} ± 0.07 | 120^{b} ± 0.07 | 108^{b} ± 0.05 | 56^{c} ± 0.03 | 587^{b} ± 0.09 |
| | paracasei DSM33375; Fr. | | | | | | |
| | sanfranciscensis DSM33378; B. | | | | | | |
| | megaterium DSM33300; B. pumilus | | | | | | |
| | DSM33297; B. licheniformis | | | | | | |
| | DSM33354 | | | | | | |
| MC15 | Lp. plantarum DSM33362, DSM33366, | 199^{d} ± 0.05 | 100^{c} ± 0.04 | 81^{c} ± 0.05 | 59^{d} ± 0.04 | 40^{d} ± 0.03 | 498^{c} ± 0.08 |
| | DSM33370; Ls. reuteri DSM33374; | | | | | | |
| | Fr. sanfranciscensis DSM33378, | | | | | | |
| | DSM33379; B. licheniformis | | | | | | |
| | DSM33354; B. subtilis DSM33353 | | | | | | |
| MC16 | Lp. plantarum DSM33363, DSM33364; | 19^{g} ± 0.03 | 11^{f} ± 0.01 | 0^{g} | 0^{e} | 0^{e} | 280^{g} ± 0.06 |
| | Lc. paracasei DSM33373; Ls. | | | | | | |
| | reuteri DSM33374; B. megaterium | | | | | | |
| | DSM33300; B. pumilus DSM33297, | | | | | | |
| | DSM33355 | | | | | | |

| | | Competitive ELISA Assay (Peptide Fragments) | | | |
|---|---|---|---|---|---|
| Strains | | 16 h | 24 h | 36 h | 48 h |
| Control | | 400^{a} ± 0.02 | 397^{a} ± 0.08 | 381^{a} ± 0.07 | 375^{a} ± 0.05 |
| MC1 | Lp. plantarum DSM33370, DSM33363, | 250^{d} ± 0.03 | 200^{e} ± 0.04 | 170^{e} ± 0.02 | 65^{g} ± 0.01 |
| | DSM33364; Lc. paracasei DSM33373; | | | | |
| | Lv. brevis DSM33377; B. pumilus | | | | |
| | DSM33297, DSM33355, DSM33301 | | | | |
| MC2 | Lp. plantarum DSM33362, DSM33367, | 226^{ef} ± 0.04 | 167^{f} ± 0.03 | 158^{e} ± 0.02 | 150^{c} ± 0.02 |
| | DSM33368; L. paracasei DSM33375; | | | | |
| | B. subtilis DSM33298; B. licheniformis | | | | |
| | DSM33354; B. megaterium DSM33300 | | | | |
| MC3 | Lp. plantarum DSM33366, DSM33369; | 272^{d} ± 0.07 | 256^{d} ± 0.04 | 244^{c} ± 0.05 | 228^{b} ± 0.02 |
| | Ls. reuteri DSM33374; Lc. paracasei | | | | |
| | DSM33376; Ped. pentosaceus DSM33371; | | | | |
| | B. megaterium DSM33356; B. subtilis | | | | |
| | DSM33353 | | | | |
| MC4 | Lp. plantarum DSM33363, DSM33364; | 233^{e} ± 0.07 | 112^{g} ± 0.05 | 0^{j} | 0^{g} |
| | Lc. paracasei DSM33373; B. subtilis | | | | |
| | DSM33298; B. pumilus DSM33301 | | | | |
| MC5 | Lv. brevis DSM33377; Ped. | 221^{e} ± 0.05 | 154^{fg} ± 0.03 | 46ⁱ ± 0.02 | 0^{g} |
| | pentosaceus DSM33371; Lp. plantarum | | | | |
| | DSM33369; B. pumilus DSM33297; | | | | |
| | B. megaterium DSM33300 | | | | |
| MC6 | Lc. paracasei DSM33375; Lp. | 245^{de} ± 0.05 | 100^{g} ± 0.08 | 79^{h} ± 0.04 | 0^{g} |
| | plantarum DSM33367, DSM33368; | | | | |
| | B. pumilus DSM33355; B. licheniformis | | | | |
| | DSM33354 | | | | |
| MC7 | Lp. plantarum DSM33370, DSM33362, | 261^{d} ± 0.05 | 150^{f} ± 0.03 | 99^{g} ± 0.04 | 78^{g} ± 0.05 |
| | DSM33366; Ls. reuteri DSM33374; | | | | |
| | B. megaterium DSM33356; B. subtilis | | | | |
| | DSM33353 | | | | |
| MC8 | Lp. plantarum DSM33363, DSM33364; | 228^{e} ± 0.06 | 218^{e} ± 0.05 | 157^{e} ± 0.06 | 0^{g} |
| | Lc. paracasei DSM33375; Ls. reuteri | | | | |
| | DSM33374; B. megaterium DSM33300; | | | | |
| | B. pumilus DSM33297 | | | | |
| MC9 | Lc. paracasei DSM33375; Lp. plantarum | 211^{f} ± 0.05 | 196^{ef} ± 0.03 | 195^{de} ± 0.07 | 152^{c} ± 0.02 |
| | DSM33367; Ls. reuteri DSM33374; | | | | |
| | B. megaterium DSM33300; B. pumilus | | | | |
| | DSM33297; B. licheniformis DSM33354 | | | | |
| MC10 | Lp. plantarum DSM33363, DSM33364, | 370^{b} ± 0.06 | 243^{de} ± 0.05 | 145^{ef} ± 0.04 | 97^{f} ± 0.03 |
| | DSM33370; Lv. brevis DSM33377; | | | | |
| | B. pumilus DSM33297; B. megaterium | | | | |
| | DSM33356 | | | | |
| MC11 | Lp. plantarum DSM33368, DSM33362, | 367^{b} ± 0.08 | 340^{b} ± 0.09 | 300^{b} ± 0.06 | 123^{de} ± 0.05 |
| | DSM33367; Lc. paracasei DSM33375; | | | | |
| | B. megaterium DSM33300; B. subtilis | | | | |
| | DSM33353 | | | | |
| MC12 | Lp. plantarum DSM33366, DSM33369; | 312^{c} ± 0.06 | 289^{c} ± 0.07 | 288^{b} ± 0.05 | 143^{cd} ± 0.03 |
| | Ls. reuteri DSM33374; Lc. paracasei | | | | |
| | DSM33376; Ped. pentosaceus | | | | |
| | DSM33371; B. pumilus DSM33297, | | | | |
| | DSM33355 | | | | |
| MC13 | Lv. brevis DSM33377; Ped. | 322^{c} ± 0.07 | 321^{b} ± 0.06 | 215^{d} ± 0.07 | 134^{d} ± 0.05 |
| | pentosaceus DSM33371; Fr. | | | | |
| | sanfranciscensis DSM33379; B. | | | | |
| | megaterium DSM33300; B. pumilus | | | | |
| | DSM33297 | | | | |
| MC14 | Lp. plantarum DSM33368; Lc. | 333^{c} ± 0.09 | 256^{d} ± 0.08 | 211^{d} ± 0.08 | 167^{c} ± 0.07 |
| | paracasei DSM33375; Fr. | | | | |
| | sanfranciscensis DSM33378; B. | | | | |
| | megaterium DSM33300; B. pumilus | | | | |
| | DSM33297; B. licheniformis | | | | |
| | DSM33354 | | | | |
| MC15 | Lp. plantarum DSM33362, DSM33366, | 318^{c} ± 0.04 | 280^{c} ± 0.03 | 256^{c} ± 0.08 | 118^{e} ± 0.05 |
| | DSM33370; Ls. reuteri DSM33374; | | | | |
| | Fr. sanfranciscensis DSM33378, | | | | |
| | DSM33379; B. licheniformis | | | | |
| | DSM33354; B. subtilis DSM33353 | | | | |
| MC16 | Lp. plantarum DSM33363, DSM33364; | 200^{f} ± 0.05 | 50^{h} ± 0.03 | 10^{j} ± 0.01 | 0^{g} |
| | Lc. paracasei DSM33373; Ls. | | | | |
| | reuteri DSM33374; B. megaterium | | | | |
| | DSM33300; B. pumilus DSM33297, | | | | |
| | DSM33355 | | | | |

^{a-j}Values with different superscript letters, in the same row, differ significantly (P < 0.05).

Based on the calculated initial amount of gluten in the reaction mixture of 7.000 ppm, regarding the residual gluten, all the mixtures were able to reduce it by at least 94% after 6 h (in comparison to a reduction of around 84% for the control), by at least 98% after 16 h and up to at least 99.1% after 48 h. Regarding gluten fragments, those were reduced by all mixtures by at least 91% after 6 h (in comparison to a reduction of around 88% for the control), by at least 95% after 16 h and up to at least 97% after 48 h.

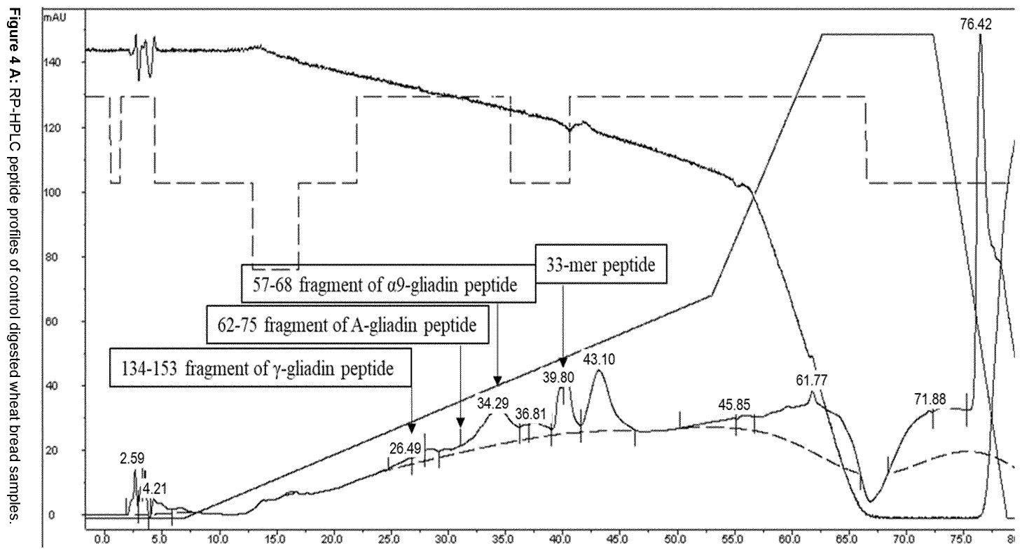

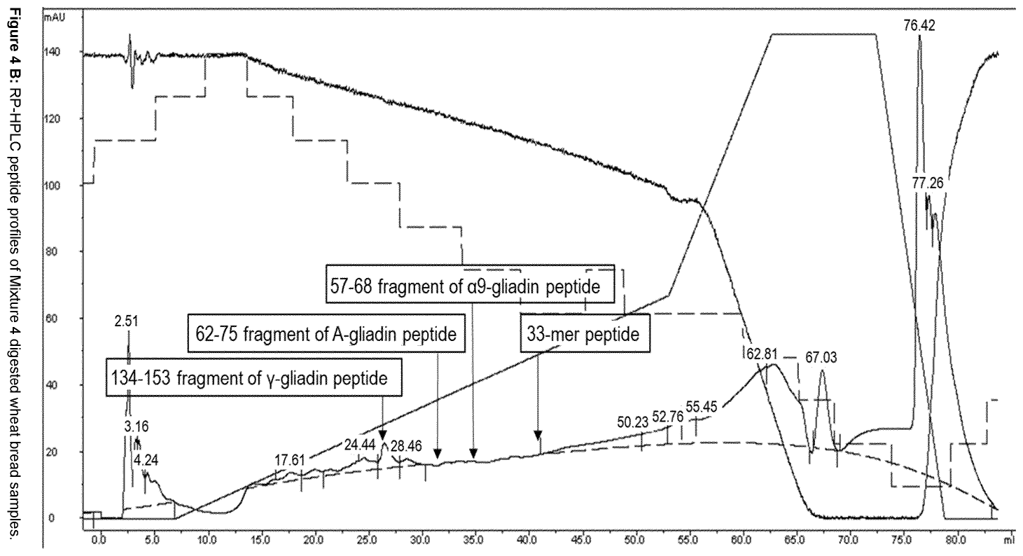

Regarding the residual gluten, the most efficient strains MC4, MC8, and MC16 were able to reduce it by at least 97% after 6 h, at least 99.8% after 16 h and up to 100% after 24 h. Regarding the gluten fragments, those were reduced by the most efficient strains MC4, MC8, and MC16 by at least 94% after 6 h, by at least 97% after 16 h, by at least 98% after 36 h and to 100% after 48 h. FIG. 4 shows RP-HPLC peptide profiles of control (panel A), Mixture 4 (panel B) and Mixture 7 (panel C) digested wheat bread samples. M4 and M7 were combined with of E1, E2, Veron PS, Veron HPP commercial enzymes. Mixture 4 led to full (93%) hydrolysis of all immunogenic peptides, whereas only partial (56%) hydrolysis was achieved by Mixture 7. In conclusion, we have found fully functional mixtures comprising only 4-7 selected strains, as compared to the more extensive mixtures disclosed in Example 3.

For exemplary microbial consortia we performed experiments with and without added commercial enzymes. The consortia alone led to strong reductions of residual as well as hydrolysed gluten, and this was further enhanced by added enzymes.

### Example 5. Assessment of Immunogenicity of Gluten Digests by Using Duodenal Explants from Celiac Disease Patients

Immunogenicity of the digests was ex vivo estimated by testing the cytokine expression in duodenal biopsy specimens from patients with celiac disease (CD). All CD patients expressed the HLA-DQ2 phenotype. CD was diagnosed according to European Society for Paediatric Gastroenterology, Hepatology, and Nutrition criteria [23]. Immediately after excision, all biopsy specimens were placed in ice-chilled culture medium (RPMI 1640; Gibco-Invitrogen, UK) and transported to the laboratory within 30 min. Duodenal biopsy specimens were cultured for 4 h using the organ tissue culture method originally described by Browning and Trier [24]. Briefly, the biopsy specimens were oriented villous side up on a stainless-steel mesh and positioned over the central well of an organ tissue culture dish (Falcon, USA). The well contained RPMI supplemented with 15% foetal calf serum (Gibco-Invitrogen) and 1% penicillin-streptomycin (Gibco-Invitrogen, UK). Dishes were placed into an anaerobic jar and incubated at 37° C.

Digested samples of control dough (positive control) (wheat bread digested without the addition of bacterial cells and microbial enzymes), Mixture 4 (wheat bread digested with the addition of live and lysed cells of L. plantarum (Lactiplantibacillus plantarum) DSM 33363 and DSM 33364, L. paracasei (Lacticaseibacillus paracasei) DSM 33373, Bacillus subtilis DSM 33298 and Bacillus pumilus DSM 33301 and E1, E2, Veron PS, Veron HPP commercial enzymes) and Mixture 7 (wheat bread digested with the addition of live and lysed cells of L. plantarum (Lactiplantibacillus plantarum) DSM 33362, and DSM 33366, Lactobacillus reuteri (Limosilactobacillus reuteri) DSM 33374, L. plantarum (Lactiplantibacillus plantarum) DSM 33370, Bacillus megaterium DSM 33356, and Bacillus subtilis DSM 33353 and E1, E2, Veron PS, Veron HPP commercial enzymes) were subjected to gliadin and glutenin polypeptide extraction and used for assessing their ability to induce cytokine expression in duodenal biopsy specimens from CD patients. Four biopsy specimens from each CD patient were cultured with culture medium under five conditions: (i) with doughs containing the Mixture 4 (wheat bread digested with the addition of live and lysed cells of L. plantarum (Lactiplantibacillus plantarum) DSM 33363 and DSM 33364, L. paracasei (Lacticaseibacillus paracasei) DSM 33373, Bacillus subtilis DSM 33298 and Bacillus pumilus DSM 33301 and E1, E2, Veron PS, Veron HPP commercial enzymes) digested for 48 h; (ii) with dough containing Mixture 7 (wheat bread digested with the addition of live and lysed cells of L. plantarum (Lactiplantibacillus plantarum) DSM 33370, DSM 33362, and DSM 33366, Lactobacillus reuteri (Limosilactobacillus reuteri) DSM 33374, Bacillus megaterium DSM 33356, and Bacillus subtilis DSM 33353 and E1, E2, Veron PS, Veron HPP commercial enzymes) digested for 48 h; (iii) with dough containing Mixture 16 (wheat bread digested with the addition of live and lysed cells of L. plantarum (Lactiplantibacillus plantarum) DSM 33363 and DSM 33364, Lactobacillus reuteri (Limosilactobacillus reuteri) DSM 33374, Bacillus megaterium DSM 33330, and Bacillus pumilus DSM 33297 and DSM 33355 and E1, E2, Veron PS, Veron HPP commercial enzymes) digested for 48 h; (iv) with control dough digested for 48 h (Control); and (v) with culture medium (RPMI 1640+gastric and intestinal juice, negative control). Biopsy specimens from each patient were rinsed and stored in RNAlater (Qiagen GmbH, Germany) at −80° C. to preserve the RNA. Total RNA was extracted from the tissues using the RNeasy minikit (Qiagen GmbH) according to the manufacturer's instructions. The concentration of mRNA was estimated by determination of the UV absorbance at 260 nm. Aliquots of total RNA (500 ng) were reverse transcribed using random hexamers, TaqMan reverse transcription reagents (Applied Biosystems, Monza, Italy), and 3.125 U/μl of MultiScribe reverse transcriptase to a final volume of 50 μl. The cDNA samples were stored at −20° C. RT-PCR was performed in 96-well plates using an ABI Prism 7500HT fast sequence detection system (Applied Biosystems). Data collection and analyses were performed using the machine software. PCR primers and fluorogenic probes for the target genes (IFN-γ, IL-2, and IL-10) and the endogenous control (gene coding for glyceraldehyde-3-phosphate dehydrogenase [GAPDH]) were purchased as a TaqMan gene expression assay and a pre-developed TaqMan assay (Applied Biosystems), respectively. The assays were supplied as a 20× mix of PCR primers and TaqMan Minor Groove Binder 6-carboxyfluorescein dye-labelled probes with a non-fluorescent quencher at the 3′ end of the probe. Two-step reverse transcription-PCR was performed using first-strand cDNA with a final concentration of 1×TaqMan gene expression assay mix and 1×TaqMan universal PCR master mix. The final reaction volume was 25 μl. Each sample was analysed in triplicate, and all experiments were repeated twice. A non-template control (RNase-free water) was included with every plate. The following thermal cycler conditions were used: 2 min at 50° C. (uracil DNA glycosylase activation), 10 min at 95° C., and 40 cycles of 15 s at 95° C. and 1 min at 60° C. Initially, a standard curve and a validation experiment were performed for each primer/probe set. Six serial dilutions (20 to 0.1 ng/μl) of IFN-γ, IL-2, or IL-10 cDNA were used as a template for each primer/probe set. A standard curve was generated by plotting the threshold cycle (CT) values against the log of the amount of input cDNA. The CT value is the PCR cycle at which an increase in reporter fluorescence above the baseline level is first detected. The average value for the target gene was normalized using an endogenous reference gene (the GAPDH gene). A healthy duodenal biopsy specimen was used to calibrate all the experiments. The levels of IFN-γ, IL-2, and IL-10 proteins secreted into the supernatant were quantified by ELISA in 96-well round-bottom plates (Tema Ricerca, Milan, Italy) according to the manufacturer's recommendations.

As expected, the duodenal biopsy specimens incubated with positive control produced significantly (P<0.05) higher expression of interleukin 2 (IL-2), interleukin 10 (IL-10) (B), and interferon gamma (IFN-γ) mRNA than the negative control (RPMI 1640+gastric and intestinal juice) (FIG. 5). Compared to negative control, the samples digested with the Mixtures 4 and 16 showed the same (P>0.05) level of IL-2, IL-10 and IFN-γ. The Mixture 7 was characterized by lower synthesis of IL-2 than the positive control, but, compared to the negative control as well as mixtures 4 and 16, by higher synthesis of IL-2. Similar trends were also found for IL-10 and IFN-γ. These results correlate nicely with the full and partial clearance of immunogenic peptides by mixture 4 and 7, respectively, as shown in FIG. 4A-C.

FIG. 5A shows concentration (ng/μl) of interleukin 2 (IL-2) in duodenal biopsy specimens from patients with CD. Control: wheat bread digested without the addition of bacterial cells and microbial enzymes; RPMI+gastric and intestinal juice: negative control; Microbial Consortium 4: wheat bread digested with the addition of live and lysed cells of L. plantarum (Lactiplantibacillus plantarum) DSM 33363 and DSM 33364, L. paracasei (Lacticaseibacillus paracasei) DSM 33373, Bacillus subtilis DSM 33298 and Bacillus pumilus DSM 33301 and E1, E2, Veron PS, Veron HPP commercial enzymes); Microbial Consortium 7: wheat bread digested with the addition of live and lysed cells of L. plantarum (Lactiplantibacillus plantarum) DSM 33362, DSM 33366 and DSM 33370, L. reuteri (Limosilactobacillus reuteri) DSM 33374, Bacillus megaterium DSM 33356, and Bacillus subtilis DSM 33353 and E1, E2, Veron PS, Veron HPP commercial enzymes; and Microbial Consortium 16: wheat bread digested with the addition of live and lysed cells of L. plantarum (Lactiplantibacillus plantarum) DSM 33363, DSM 33364, L. paracasei (Lacticaseibacillus paracasei) DSM 33373, L. reuteri (Limosilactobacillus reuteri) DSM 33374, Bacillus megaterium DSM 33330, Bacillus pumilus DSM 33297, DSM 33355. CD1 to CD10, duodenal biopsy specimens from celiac patients.

FIG. 5 B shows concentration (ng/μl) of interleukin 10 (IL-10) in duodenal biopsy specimens from patients with CD. Samples and microbial consortia are equivalent to FIG. 5A.

FIG. 5 C shows concentration (ng/μl) of interferon gamma (IFN-γ) in duodenal biopsy specimens from patients with CD. Samples and microbial consortia are equivalent to FIG. 5A.

The findings of this invention provide evidence that the selected combinations of probiotic bacterial strains have the potential to improve the digestion of gluten in gluten-sensitive patients and to hydrolyse immunogenic peptides during gastrointestinal digestion, which decreases gluten toxicity for gluten-sensitive patients in general, and for CD patients particularly.

The following strain mixtures were identified with the present screening process according to the present invention:
L. plantarum (Lactiplantibacillus plantarum) DSM 33370, DSM 33363, DSM 33364, DSM 33365; L. paracasei (Lacticaseibacillus paracasei) DSM 33373; L. brevis (Levilactobacillus brevis) DSM 33377; Bacillus pumilus DSM 33297, DSM 33355, Bacillus licheniformis DSM 33354, Bacillus megaterium DSM 33300 and Bacillus subtilis DSM 33353, or
L. plantarum (Lactiplantibacillus plantarum) DSM 33362, DSM 33367, DSM 33368; L. paracasei (Lacticaseibacillus paracasei) DSM 33375; L. sanfranciscensis (Fructilactobacillus sanfranciscensis) DSM 33379; Bacillus pumilus DSM 33301, Bacillus megaterium DSM 33300, DSM 33356, Bacillus subtilis DSM 33298 and DSM 33353, or
L. plantarum (Lactiplantibacillus plantarum) DSM 33366 and DSM 33369, Lactobacillus reuteri (Limosilactobacillus reuteri) DSM 33374; L. paracasei (Lacticaseibacillus paracasei) DSM 33376; Pediococcus pentosaceus DSM 33371; L. sanfranciscensis (Fructilactobacillus sanfranciscensis) DSM 33378; Bacillus licheniformis DSM 33354, Bacillus pumilus DSM 33301, Bacillus megaterium DSM 33300, DSM 33356 and Bacillus subtilis DSM 33298.
L. plantarum (Lactiplantibacillus plantarum) DSM 33370, DSM 33363 and DSM 33364, L. paracasei (Lacticaseibacillus paracasei) DSM 33373 L. brevis (Levilactobacillus brevis) DSM 33377, Bacillus pumilus DSM 33297, DSM 33355, DSM 33301;
L. plantarum (Lactiplantibacillus plantarum) DSM 33362 and DSM 33367, DSM 33368, L. paracasei (Lacticaseibacillus paracasei) DSM 33375, Bacillus subtilis DSM 33298, Bacillus licheniformis DSM 33354, and Bacillus megaterium DSM 33300;
L. plantarum (Lactiplantibacillus plantarum) DSM 33366, DSM 33369, Lactobacillus reuteri (Limosilactobacillus reuteri) DSM 33374, L. paracasei (Lacticaseibacillus paracasei) DSM 33376, Pediococcus pentosaceus DSM 33371, Bacillus megaterium DSM 33356, and Bacillus subtilis DSM 33353;
L. plantarum (Lactiplantibacillus plantarum) DSM 33363 and DSM 33364, L. paracasei (Lacticaseibacillus paracasei) DSM 33373, Bacillus subtilis DSM 33298 and Bacillus pumilus DSM 33301;
L. brevis (Levilactobacillus brevis) DSM 33377, Pediococcus pentosaceus DSM 33371, L. plantarum (Lactiplantibacillus plantarum) DSM 33369, Bacillus pumilus DSM 33297 and Bacillus megaterium DSM 33300;
L. paracasei (Lacticaseibacillus paracasei) DSM 33375, L. plantarum (Lactiplantibacillus plantarum) DSM 33367, DSM 33368; Bacillus pumilus DSM 33355, and Bacillus licheniformis DSM 33354;
L. plantarum (Lactiplantibacillus plantarum) DSM 33370, DSM 33362, and DSM 33366, Lactobacillus reuteri (Limosilactobacillus reuteri) DSM 33374, Bacillus megaterium DSM 33356, and Bacillus subtilis DSM 33353.
L. plantarum (Lactiplantibacillus plantarum) DSM 33363, DSM 33364, L. paracasei (Lacticaseibacillus paracasei) DSM 33375, L. reuteri (Limosilactobacillus reuteri) DSM 33374, B. megaterium DSM 33300, B. pumilus DSM 33297;
L. paracasei (Lacticaseibacillus paracasei) DSM 33375, L. plantarum (Lactiplantibacillus plantarum) DSM 33367, L. reuteri (Limosilactobacillus reuteri) DSM 33374, B. megaterium DSM 33300, B. pumilus DSM 33297, B. licheniformis DSM 33354;
L. plantarum (Lactiplantibacillus plantarum) DSM 33363, DSM 33364, DSM 33370, L. brevis (Levilactobacillus brevis) DSM 33377, B. pumilus DSM 33297, Bacillus megaterium DSM 33356;
L plantarum (Lactiplantibacillus plantarum) DSM 33362, DSM 33367, DSM 33368, L. paracasei (Lacticaseibacillus paracasei) DSM 33375, B. megaterium DSM 33300, B. subtilis DSM 33353;
L. plantarum (Lactiplantibacillus plantarum) DSM 33366, DSM 33369, L. reuteri (Limosilactobacillus reuteri) DSM 33374, L. paracasei (Lacticaseibacillus paracasei) DSM 33376, P. pentosaceus DSM 33371, B. pumilus DSM 33297, DSM 33355;
L. brevis (Levilactobacillus brevis) DSM 33377, P. pentosaceus DSM 33371, L. sanfranciscensis (Fructilactobacillus sanfranciscensis) DSM 33379, B. megaterium DSM 33300, B. pumilus DSM 33297;
L. plantarum (Lactiplantibacillus plantarum) DSM 33368, L. paracasei (Lacticaseibacillus paracasei) DSM 33375, L. sanfranciscensis (Fructilactobacillus sanfranciscensis) DSM 33378, B. megaterium DSM 33300, B. pumilus DSM 33297, B. licheniformis DSM 33354;
L. plantarum (Lactiplantibacillus plantarum) DSM 33362, DSM 33366, DSM 33370, L. reuteri (Limosilactobacillus reuteri) DSM 33374, L. sanfranciscensis (Fructilactobacillus sanfranciscensis) DSM 33378, DSM 33379, B. licheniformis DSM 33354, B. subtilis DSM 33353;
L. plantarum (Lactiplantibacillus plantarum) DSM 33363, DSM 33364, L. paracasei (Lacticaseibacillus paracasei) DSM 33373, L. reuteri (Limosilactobacillus reuteri) DSM 33374, B. megaterium DSM 33300, B. pumilus DSM 33297, DSM 33355.

The preferred combinations were:
L. plantarum (Lactiplantibacillus plantarum) DSM 33363 and DSM 33364, L. paracasei (Lacticaseibacillus paracasei) DSM 33373, Bacillus subtilis DSM 33298, and Bacillus pumilus DSM 33301, or
L. plantarum (Lactiplantibacillus plantarum) DSM 33363 and DSM 33364, L. paracasei (Lacticaseibacillus paracasei) DSM 33375, Lactobacillus reuteri (Limosilactobacillus reuteri) DSM 33374, Bacillus megaterium DSM 33300, and Bacillus pumilus DSM 33297, or
L. plantarum (Lactiplantibacillus plantarum) DSM 33363 and DSM 33364, L. paracasei (Lacticaseibacillus paracasei) DSM 33373, Lactobacillus reuteri (Limosilactobacillus reuteri) DSM 33374, Bacillus megaterium DSM 33300, Bacillus pumilus DSM 33297, Bacillus pumilus DSM 33355.

### REFERENCES

1. Sapone A, Bai J C, Ciacci C, Dolinsek J, Green P H, Hadjivassiliou M, Kaukinen K, Rostami K, Sanders D S, Schumann M et al: Spectrum of gluten-related disorders: consensus on new nomenclature and classification. BMC Med 2012, 10:13.
2. Niland B, Cash B D: Health Benefits and Adverse Effects of a Gluten-Free Diet in Non-Celiac Disease Patients. Gastroenterol Hepatol (N Y) 2018, 14(2):82-91.
3. Lis D M, Stellingwerff T, Shing C M, Ahuja K D, Fell J W: Exploring the popularity, experiences, and beliefs surrounding gluten-free diets in nonceliac athletes. Int J Sport Nutr Exerc Metab 2015, 25(1):37-45.
4. Wild D, Robins G G, Burley V J, Howdle P D: Evidence of high sugar intake, and low fibre and mineral intake, in the gluten-free diet. Aliment Pharmacol Ther 2010, 32(4):573-581.
5. Thompson T, Dennis M, Higgins L A, Lee A R, Sharrett M K: Gluten-free diet survey: are Americans with coeliac disease consuming recommended amounts of fibre, iron, calcium and grain foods? J Hum Nutr Diet 2005, 18(3):163-169.
6. Larretxi I, Simon E, Benjumea L, Miranda J, Bustamante M A, Lasa A, Eizaguirre F J, Churruca I: Gluten-free-rendered products contribute to imbalanced diets in children and adolescents with celiac disease. Eur J Nutr 2018.
7. De Palma G, Nadal I, Collado M C, Sanz Y: Effects of a gluten-free diet on gut microbiota and immune function in healthy adult human subjects. Br J Nutr 2009, 102(8):1154-1160.
8. Galipeau H J, McCarville J L, Huebener S, Litwin O, Meisel M, Jabri B, Sanz Y, Murray J A, Jordana M, Alaedini A et al: Intestinal microbiota modulates gluten-induced immunopathology in humanized mice. Am J Pathol 2015, 185(11):2969-2982.
9. Caminero A, Galipeau H J, McCarville J L, Johnston C W, Bernier S P, Russell A K, Jury J, Herran A R, Casqueiro J, Tye-Din J A et al: Duodenal Bacteria From Patients With Celiac Disease and Healthy Subjects Distinctly Affect Gluten Breakdown and Immunogenicity. Gastroenterology 2016, 151(4):670-683.
10. Francavilla R, Ercolini D, Piccolo M, Vannini L, Siragusa S, De Filippis F, De Pasquale I, Di Cagno R, Di Toma M, Gozzi G et al: Salivary microbiota and metabolome associated with celiac disease. Appl Environ Microbiol 2014, 80(11):3416-3425.
11. Krishnareddy S, Stier K, Recanati M, Lebwohl B, Green P H: Commercially available glutenases: a potential hazard in coeliac disease. Therap Adv Gastroenterol 2017, 10(6):473-481.
12. Shan L, Marti T, Sollid L M, Gray G M, Khosla C: Comparative biochemical analysis of three bacterial prolyl endopeptidases: implications for coeliac sprue. Biochem J 2004, 383(Pt 2):311-318.
13. Rashmi B S, Gayathri D: Molecular characterization of gluten hydrolysing Bacillus sp. and their efficacy and biotherapeutic potential as probiotics using Caco-2 cell line. J Appl Microbiol 2017, 123(3):759-772.
14. Phromraksa P, Nagano H, Boonmars T, Kamboonruang C: Identification of proteolytic bacteria from thai traditional fermented foods and their allergenic reducing potentials. J Food Sci 2008, 73(4):M189-195.
15. Fernandez-Feo M, Wei G, Blumenkranz G, Dewhirst F E, Schuppan D, Oppenheim F G, Helmerhorst E J: The cultivable human oral gluten-degrading microbiome and its potential implications in coeliac disease and gluten sensitivity. Clin Microbiol Infect 2013, 19(9):E386-394.
16. Francavilla R, De Angelis M, Rizzello C G, Cavallo N, Dal Bello F, Gobbetti M: Selected Probiotic Lactobacilli Have the Capacity To Hydrolyze Gluten Peptides during Simulated Gastrointestinal Digestion. Appl Environ Microbiol 2017, 83(14).
17. Herran A R, Perez-Andres J, Caminero A, Nistal E, Vivas S, Ruiz de Morales J M, Casqueiro J: Gluten-degrading bacteria are present in the human small intestine of healthy volunteers and celiac patients. Res Microbiol 2017, 168(7):673-684.
18. Caminero A, Herran A R, Nistal E, Perez-Andres J, Vaquero L, Vivas S, Ruiz de Morales J M, Albillos S M, Casqueiro J: Diversity of the cultivable human gut microbiome involved in gluten metabolism: isolation of microorganisms with potential interest for coeliac disease. FEMS Microbiol Ecol 2014, 88(2):309-319.
19. Fernandez M F, Boris S, Barbes C: Probiotic properties of human lactobacilli strains to be used in the gastrointestinal tract. J Appl Microbiol 2003, 94(3):449-455.
20. Zarate G, Chaia A P, Gonzalez S, Oliver G: Viability and beta-galactosidase activity of dairy propionibacteria subjected to digestion by artificial gastric and intestinal fluids. J Food Prot 2000, 63(9):1214-1221.
21. De Angelis M, Siragusa S, Berloco M, Caputo L, Settanni L, Alfonsi G, Amerio M, Grandi A, Ragni A, Gobbetti M: Selection of potential probiotic lactobacilli from pig feces to be used as additives in pelleted feeding. Res Microbiol 2006, 157(8):792-801.
22. Weiss W, Vogelmeier C, Gorg A: Electrophoretic characterization of wheat grain allergens from different cultivars involved in bakers' asthma. Electrophoresis 1993, 14(8):805-816.
23. Bradford M M: A rapid and sensitive method for the quantitation of microgram quantities of protein utilizing the principle of protein-dye binding. Anal Biochem 1976, 72:248-254.
24. Valdes I, Garcia E, Llorente M, Mendez E: Innovative approach to low-level gluten determination in foods using a novel sandwich enzyme-linked immunosorbent assay protocol. Eur J Gastroenterol Hepatol 2003, 15(5):465-474.
25. Browning T H, Trier J S: Organ culture of mucosal biopsies of human small intestine. J Clin Invest 1969, 48(8):1423-1432.
26. of ES: Revised criteria for diagnosis of coeliac disease. Report of Working Group of European Society of Paediatric Gastroenterology and Nutrition. Arch Dis Child 1990, 65(8):909-911.

1. A process to identify a consortium of probiotic strains for promoting a degradation of gluten and gluten-derived peptides (epitopes) comprising:
1) providing a library of at least 10 probiotic bacterial strains;
2) incubating the probiotic bacterial strains of 1) to simulated gastric (pH 1-4) conditions for at least 30 minutes and intestinal conditions (pH 5.5-8.5) for at least 30 minutes and selecting strains with less than 2 log loss of CFU after simulated gastric and intestinal conditions;
3) determining proteinase activities of the strains selected in 2) towards gluten and selecting strains with capability to decrease an initial gluten level of at least 5000 ppm by 10 to 70%;
4) determining activities of peptidases aminopeptidase type N (PepN); PepI, PepO, Prolyl endopeptidyl peptidase (PEP); PepX, and PepQ peptide hydrolase of the strains selected in 3) and selecting strains with peptidase activity of at least 1 U/g for at least one of these peptidases;
5) combining at least 2 of the strains selected in 4) to a consortium of probiotic strains with activities of the peptidases PepN, PepI, PepO, PepX and PepQ of at least 1 U/g for each peptidase;
6) determining peptidase activities of the consortium of 5) with peptidase activity towards the 12-mer peptide QLQPFPQPQLPY (Seq-ID No 1), the 14-mer peptide PQPQLPYPQPQSFP (Seq-ID No 2), the 20-mer peptide QQLPQPQQPQQSFPQQQRPF (Seq-ID No 3), and the 33-mer peptide LQLQPFPQPQLPYPQPQLPYPQPQLPYPQPQPF (Seq-ID No 4)) and selecting a of consortium with a peptidase activity to degrade all four epitopes by more than 50%; and
7) determining peptidase activity for the consortium selected in 6) for the hydrolysis of gluten with a starting concentration of at least 5000 ppm gluten under simulated gastric (pH 1-4) conditions for at least 30 minutes and intestinal conditions (pH 5.5-8.5) for at least 30 minutes and selecting a consortium that reduces an initial gluten level of at least 5000 ppm to a concentration of hydrolyzed and residual gluten to less than 200 ppm.

2. The process according to claim 1, further comprising:
8) determining hydrolysis of gluten during wheat bread digestion (1-100 gr of wheat bread) by the consortium of strains selected in 6) under simulated gastrointestinal conditions and selection of strains with a degradation capacity of the gluten content in wheat bread during 6-24 hours to less than 20 ppm and absence of gluten-derived epitopes (the 12-mer peptide, the 14-mer peptide, the 20-mer peptide and the 33-mer peptide) after 180 min of simulated intestinal digestion;
9) determining immunogenicity of the consortium of strains selected in 7) by using small intestinal tissue explants from CD patients by determining the expression of the cytokines Interleukin 2 (IL-2), interleukin 10 (IL-10), and interferon gamma (IFN-γ) after an incubation of 6-48 h under gastro-intestinal conditions and selection of strains with an immunogenicity of not more than the negative control.

3. The process according to claim 1, wherein the gastric conditions for 2) and 7) include incubation of strains at pH 1-4 for a time of between 30 minutes and 300 minutes at a temperature between 35° C. and 39° C. in simulated gastric fluid containing pepsin (0.5-6 g/l) and the intestinal conditions for 2) and 7) include incubation of strains at pH 5.5-pH 8.5 for a time of between 30 minutes and 300 minutes at a temperature between 35° C. and 39° C. in simulated intestinal fluid containing pancreatin (0.02-0.6% w/v) and bile salts (0.05-0.6%).

4. The process according to claim 1, wherein the activities of peptidases aminopeptidase type N (PepN); PepI, PepO, Prolyl endopeptidyl peptidase (PEP); PepX, and PepQ peptide hydrolase in 4) are determined using strains at a density between 7.0 and 11.0 log CFU/ml in the form of viable cells or cytoplasmic extracts thereof with peptide substrates with amino acid sequences suitable for detection of aminopeptidase type N (PepN); PepI, PepO, Prolyl endopeptidyl peptidase (PEP); PepX, and PepQ peptide hydrolase activities.

5. The process according to claim 1, wherein the peptidase activities in 5) are determined by using viable cells or cytoplasmic extracts thereof in buffered media (pH 6.0-9.0) at 35° C.-39° C. for 1-12 h and the strains with a degradation capacity of all four epitopes of more than 95% are selected.

6. The process according to claim 2, wherein the simulated gastrointestinal conditions in 8) include incubating the strains selected in 6) at a density between 7.0 and 11.0 log CFU/ml, their cytoplasm and/or Bacillus proteases at pH 2-4 for a time of between 30 minutes and 300 minutes at a temperature between 36.5° C. and 37° C. in simulated gastric fluid containing pepsin (0.5-6 g/l) and of incubating the strains selected in 6), their cytoplasm and/or Bacillus proteases at pH 7.0-pH 8.5 for a time of between 30 minutes and 48 hours at a temperature between 36.5° C. and 37° C. in simulated intestinal fluid containing pancreatin (0.02-0.6% w/v) and bile salts (0.05-0.6%).

7. The process according to claim 1, wherein the bacterial strains are derived from one or more of soil, cereals (wheat, ryes, barley), cereal processing, sourdough, feces from humans, pigs, dogs, cats, rats, and mice, and gastrointestinal tract specimens from humans, pigs, dogs, cats, rats, and mice.

8. The process according to claim 1, wherein the bacterial strains are one or more genera selected from the group consisting of Lactobacillus, Bacillus, Pediococcus and Weissella.
